(19) United States
(12) Patent Application Publication
(10) Pub. No.: US 2023/0383235 A1
(43) Pub. Date: Nov. 30, 2023

(54) BIOPARTICLE SORTING APPARATUS AND INFORMATION PROCESSING APPARATUS

(71) Applicant: Sony Group Corporation, Tokyo (JP)

(72) Inventors: Ryu Sukigara, Kanagawa (JP); Koichiro Sato, Kanagawa (JP); Muneaki Osawa, Chiba (JP); Shinichi Yoshida, Kanagawa (JP); Yudai Yanashita, Kanagawa (JP); Marcaurele Brun, Tokyo (JP); Hazime Matsuda, Kanagawa (JP)

(73) Assignee: Sony Group Corporation, Tokyo (JP)

(21) Appl. No.: 18/030,305

(22) PCT Filed: Aug. 30, 2021
(86) PCT No.: PCT/JP2021/031734

§ 371 Date: Apr. 5, 2023

Related U.S. Application Data
(60) Provisional application No. 63/090,676, filed on Oct. 12, 2020.

(51) Int. Cl. C12M 1/36; C12M 1/00

(52) U.S. Cl. CPC C12M 41/48; C12M 47/04

(57) ABSTRACT

A main objective of the present disclosure is to improve the operability of a bioparticle sorting apparatus that accepts input of various items of data according to a touch operation.

The present disclosure provides a bioparticle sorting apparatus including a display unit that is capable of touch input and displays a processing condition setting screen for accepting input of operation control data pertaining to a sorting process for a bioparticle-containing sample, the processing condition setting screen including a first display area for displaying an operation flow for setting a sorting process condition for the bioparticle-containing sample and a second display area for displaying measurement data pertaining to the bioparticle-containing sample, the first display area being disposed in an end section of the processing condition setting screen.

### TECHNICAL FIELD

The present disclosure pertains to a bioparticle sorting apparatus and an information processing apparatus. In more detail, the present disclosure pertains to a bioparticle sorting apparatus and an information processing apparatus that display a screen pertaining to control and/or execution of a bioparticle sorting process.

### BACKGROUND ART

In flow cytometry, light originating from many types of fluorochromes is analyzed in a multidimensional fashion. For this analysis, many graphs and/or tables of statistics are displayed on a screen belonging to an information processing apparatus, and, for example, setting or adjustment of a gate is performed. In addition, using a result of the analysis to sort a desired bioparticle is also performed. The apparatus that executes this sorting is also referred to as a cell sorter. In addition, in recent years, an apparatus that sorts bioparticles in a closed space has also been proposed, and this apparatus is also referred to as a closed sorter.

In relation to closed sorters, several proposals have been made thus far. For example, the following PTL 1 discloses a microparticle sorting apparatus that includes a determination unit that, according to light arising by application of light onto microparticles flowing through a flow path, determines whether to sort the microparticles, in which the determination unit performs a primary sorting determination to determine, according to characteristics of the light, whether the microparticles belong to any one of two or more different microparticle populations, and then performs a secondary sorting determination to determine, according to a particle composition ratio designated for the two or more different microparticle populations, whether to sort the microparticles determined to belong to any one of the microparticle populations in the primary sorting determination.

### CITATION LIST

### Patent Literature

### [PTL 1]

Japanese Patent Laid-open No. 2020-076736

### SUMMARY

### Technical Problem

A sorting process condition setting such as a gate setting is performed for a sorting process by a bioparticle sorting apparatus. This setting is performed via various items of data displayed on a screen, as described above. It is often the case that advanced specialized knowledge (for example, knowledge pertaining to bioparticles, knowledge pertaining to a sorting apparatus, etc.) is required to perform this setting. A user interface for an apparatus such as a flow cytometer or a cell sorter that has been commercially available thus far has been designed on a premise that a user has such specialized knowledge.

However, research, development, and manufacturing pertaining to a cell therapeutic agent, for example, is performed in a clean room in order to avoid contamination. Accordingly, for example, in a case of using a bioparticle sorting apparatus such as a closed sorter in a clean room, as illustrated in FIG. 1, for example, it is envisioned that a user U operates a bioparticle sorting apparatus A in a state where the user U is wearing clean room work clothing and gloves. The gloves are laboratory gloves made from a material such as latex, for example. In a case where such gloves are worn on hands, fine operation is often difficult. In relation to a touch panel, for example, fine operation is difficult in a state where such gloves are worn. Accordingly, it is desirable to provide a user interface that is easy to use or easy to operate in such a state.

Accordingly, a main objective of the present disclosure is to improve the operability of a bioparticle sorting apparatus that accepts input of various items of data according to a touch operation.

### Solution to Problem

The present disclosure provides a bioparticle sorting apparatus that includes a display unit that is capable of touch input and displays a processing condition setting screen for accepting input of operation control data pertaining to a sorting process for a bioparticle-containing sample, in which the processing condition setting screen includes a first display area for displaying an operation flow for setting a sorting process condition for the bioparticle-containing sample and a second display area for displaying measurement data pertaining to the bioparticle-containing sample, and the first display area is disposed in an end section of the processing condition setting screen.

In addition, the present disclosure also provides an information processing apparatus that includes a display unit that is capable of touch input and displays a processing condition setting screen for accepting input of operation control data pertaining to a sorting process for a bioparticle-containing sample, in which the processing condition setting screen includes a first display area for displaying an operation flow for setting a sorting process condition for the bioparticle-containing sample and a second display area for displaying measurement data pertaining to the bioparticle-containing sample, and the first display area is disposed in an end section of the processing condition setting screen.

### BRIEF DESCRIPTION OF DRAWINGS

FIG. 1 is a schematic view that illustrates an example of a situation in which a bioparticle sorting apparatus is being operated.

FIG. 2A is a view that illustrates an example of a configuration of a biological sample analysis apparatus according to the present disclosure.

FIG. 2B is an example of a block view of the bioparticle sorting apparatus according to the present disclosure.

FIG. 3 is an example of a flow chart for processing performed by the bioparticle sorting apparatus according to the present disclosure.

FIG. 4 is a view that illustrates an example of a mode selection screen.

FIG. 5 is a view that illustrates an example of an identification information input screen.

FIG. 6 is a view that illustrates an example of a processing condition setting screen.

FIG. 7 is a view that illustrates an example of the processing condition setting screen.

FIG. 8 is a view that illustrates an example of a first display area.

FIG. 9 is a view that illustrates an example of a second display area.

FIG. 10A is a view for describing a tab area.

FIG. 10B is a view for describing the tab area.

FIG. 10C is a view for describing the tab area.

FIG. 11 is a view that illustrates an example of a processing flow in a first display mode.

FIG. 12 is a view that illustrates an example of a processing condition setting screen.

FIG. 13 is a view that illustrates an example of the processing condition setting screen.

FIG. 14 is a view that illustrates an example of a setting information display area.

FIG. 15A is a view that illustrates an example of a window displayed by a matrix display button being selected.

FIG. 15B is a view that illustrates an example of a situation for adjustment of each correction coefficient in a correction matrix.

FIG. 16 is a view that illustrates an example of a window displayed by a gate tree display button being selected.

FIG. 17 is a view that illustrates an example of a toolbox displayed by a worksheet toolbox display button being selected.

FIG. 18 is a view that illustrates an example of a toolbox displayed by a plot toolbox display button being selected.

FIG. 19 is a view that illustrates an example of a toolbox displayed by a gate toolbox display button being selected.

FIG. 20 is a view that illustrates an example of a screen for controlling a sorting process.

FIG. 21 is a view that illustrates an example of a template property area.

FIG. 22 is a view that illustrates an example of a screen for an aliquot setting area.

FIG. 23 is a view that illustrates an example of an operator-directed instruction data input area.

FIG. 24 is a view that illustrates an example of a processing execution operation screen.

FIG. 25 is a view that illustrates an example of a fourth display area.

FIG. 26 is a view that illustrates an example of a processing execution operation screen.

FIG. 27 is a view that illustrates an example of the processing execution operation screen.

FIG. 28A is a view that illustrates an example of the processing execution operation screen.

FIG. 28B is a view that illustrates an example of the processing execution operation screen.

FIG. 29 is a view that illustrates an example of the processing execution operation screen.

FIG. 30 is a view that illustrates an example of a window displayed at a time of aliquot obtainment.

FIG. 31 is a view that illustrates an example of a bioparticle sorting system according to the present disclosure.

FIG. 32A is a view that illustrates an example of an operation panel.

FIG. 32B is a view that illustrates an example of an operation panel.

FIG. 33A is a view that illustrates an example of slider bars.

FIG. 33B is a view that illustrates an example of slider bars.

FIG. 33C is a view that illustrates an example of slider bars.

FIG. 33D is a view that illustrates an example of slider bars.

FIG. 34A is a view for describing an enlarged image displayed in a gate adjustment.

FIG. 34B is a view for describing an enlarged image displayed in a gate adjustment.

### DESCRIPTION OF EMBODIMENTS

Description is given below regarding a suitable aspect for implementing the present disclosure. Note that embodiments described below indicate embodiments that are representative of the present disclosure, and the scope of the present disclosure is not limited to these embodiments. Note that description of the present disclosure is given in the following order.
1. General overview of the present disclosure
2. First embodiment (bioparticle sorting apparatus) of the present disclosure
(1) Apparatus configuration example
(2) Example of processing
(2-1) Selection of display mode
(2-2) First display mode
(2-2-1) Example of configuration of screen displayed in first display mode
(2-2-2) Example of processing in first display mode
(2-3) Second display mode
(2-3-1) Example of configuration of screen displayed in second display mode
(2-3-2) Example of processing in second display mode
(3) Variations
3. Second embodiment (bioparticle sorting system) of the present disclosure

### 1. General Overview of the Present Disclosure

As described above, in a case of operating a bioparticle sorting apparatus in a clean room, a user who operates the apparatus is typically wearing gloves. In a case where such gloves are worn on hands, fine operation is often difficult. In relation to a touch panel for example, fine operation is difficult in a state where such gloves are worn. Accordingly, it is desirable to provide a user interface that is easy to use or easy to operate in such a state.

In addition, a sorting process condition setting for a sorting process by a bioparticle sorting apparatus is, as described above, performed via various items of data displayed on a screen, and there are many cases where advanced specialized knowledge is required to set a sorting process condition. Accordingly, it is considered that a user interface in the related art is difficult to use appropriately for a user such as a worker or an operator mentioned above. Hence, it is desirable to provide a user interface that is easy to use or easy to operate for such a user.

The present disclosure provides a bioparticle sorting apparatus that includes a display unit that is capable of touch input and displays a processing condition setting screen for accepting input of operation control data pertaining to a sorting process for a bioparticle-containing sample. Here, the processing condition setting screen may include a first display area for displaying an operation flow for setting a sorting process condition for the bioparticle-containing sample and a second display area for displaying measurement data pertaining to the bioparticle-containing sample. In addition, the first display area may be disposed in an end section of the processing condition setting screen.

The bioparticle sorting apparatus that causes the display unit to display the processing condition setting screen is suitable for an operation according to touch input. This apparatus is easy to use, even for a user who is wearing gloves, for example.

Provided is a bioparticle sorting apparatus having a first display mode for accepting input of operation control data pertaining to a bioparticle sorting process and a second display mode in which a processing execution operation screen is displayed. In one embodiment of the present disclosure, this bioparticle sorting apparatus may be configured to operate in the first display mode or the second display mode in reference to identification information.

The bioparticle sorting apparatus according to the present disclosure may be configured to operate in any one of two display modes, for example. These two display modes are, for example, a first display mode for accepting input of operation control data pertaining to a bioparticle sorting process and a second display mode in which a processing execution operation screen is displayed.

The bioparticle sorting apparatus may be configured to cause the display unit to display the processing condition setting screen in the first display mode.

A sorting process condition setting such as a gate setting is performed for a sorting process by a bioparticle sorting apparatus. This setting is performed via various items of data displayed on a screen, as described above. It is often the case that advanced specialized knowledge (for example, knowledge pertaining to bioparticles, knowledge pertaining to a sorting apparatus, etc.) is required to perform this setting. A user interface for an apparatus such as a flow cytometer or a cell sorter that has been commercially available thus far has been designed on a premise that a user has such specialized knowledge.

However, for example, for the manufacture of a cell therapeutic agent, etc., in order to sort a large amount of desired cells with use of a bioparticle sorting apparatus, it is envisioned that the setting itself of a sorting process condition will be performed by a user (for example, a researcher, developer, or the like who has the abovementioned advanced specialized scientific knowledge) who is familiar with use of the abovementioned various items of data, while an actual sorting operation will be performed by a user (for example, a worker, operator, or the like in a factory for manufacturing the cell therapeutic agent) who, in comparison to the abovementioned apparatus user, is not familiar with use of the various items of data.

By having the two display modes as described above, it is possible, for example, for a researcher or a developer to use the first display mode to input operation control data and for a worker or an operator to execute a sorting process by the bioparticle sorting apparatus via the processing execution operation screen in the second mode. Accordingly, it is possible to present respectively different user interfaces to a researcher or a developer as well as a worker or an operator, and it is possible to provide work environments that are suitable for respective use, for example.

For example, in the first display mode, a researcher or a developer inputs, via the processing condition setting screen, the abovementioned operation control data (for example, work procedure information or the like) pertaining to a sorting process operation that is to be executed by a worker or an operator. In addition, in the second display mode, a processing execution operation screen generated in reference to the operation control data is presented to the operator. As a result, the researcher or developer can easily create an operator-directed instruction, such as a correct work procedure and/or important points for each sorting process. Further, the operator can follow the instruction displayed on the processing execution operation screen and execute an appropriate sorting process while following a designated work procedure and referring to important points.

In the present specification, a user who operates a bioparticle sorting apparatus in the first display mode is referred to as a “first user,” and a user who operates the bioparticle sorting apparatus in the second display mode is referred to as a “second user.”

The first user may be a user who designs and/or develops a sorting process that is to be executed in the second display mode. The first user is, for example, the abovementioned researcher, developer, or the like.

The second user may be a user who executes a sorting process by operating the bioparticle sorting apparatus in accordance with an instruction inputted in the first display mode. The second user is, for example, the abovementioned operator, worker, or the like.

Below, an example of a configuration of an apparatus is described first, and an example of operation by a bioparticle sorting apparatus according to the present disclosure is described next.

### 2. First Embodiment (Bioparticle Sorting Apparatus) of the Present Disclosure

### (1) Apparatus Configuration Example

A bioparticle sorting apparatus according to the present disclosure may be configured as a biological sample analysis apparatus as described below.

FIG. 2A illustrates an example of a configuration of a biological sample analysis apparatus according to the present disclosure. A biological sample analysis apparatus 6100 illustrated in FIG. 2A includes a light application unit 6101 that applies light onto a biological sample S that flows in a flow path C, a detection unit 6102 that detects light arising by light being applied onto the biological sample S, and an information processing unit 6103 that processes information pertaining to light detected by the detection unit. It is possible to give a flow cytometer and an imaging cytometer as examples of the biological sample analysis apparatus 6100. The biological sample analysis apparatus 6100 may include a sorting unit 6104 that sorts specific bioparticles P that are within a biological sample. It is possible to give a cell sorter as an example of the biological sample analysis apparatus 6100 that includes the sorting unit.

### (Biological Sample)

The biological sample S may be a liquid sample that includes bioparticles. These bioparticles are cells or acellular bioparticles, for example. The abovementioned cells may be living cells, and it possible to give, as more specific examples thereof, blood cells such as red blood cells or white blood cells and germ cells such as sperm or fertilized eggs. In addition, the abovementioned cells may be directly collected from a specimen such as whole blood, or may be cultured cells obtained after cultivation. As the abovementioned acellular bioparticles, it is possible to give extracellular vesicles, in particular, exosomes, micro vesicles, and the like. The abovementioned bioparticles may be labeled by one or more labeling substances (for example, a colorant (specifically, a fluorochrome), a fluorochrome-labeled antibody, etc.). Note that particles other than bioparticles may be analyzed by the biological sample analysis apparatus according to the present disclosure, and beads or the like may be analyzed for calibration, etc.

### (Flow Path)

The flow path C is configured such that the biological sample S flows. In particular, the flow path C can be configured such that a flow in which bioparticles included in the abovementioned biological sample are lined up approximately in a row is formed. A flow path structure that includes the flow path C may be designed such that a laminar flow is formed. In particular, this flow path structure is designed such that a laminar flow in which a flow for a biological sample (sample flow) is enclosed by a sheath liquid flow is formed. A design for this flow path structure may be selected, as appropriate, by a person skilled in the art, or something known may be employed. The flow path C may be formed in a flow path structure (flow channel structure) such as a microchip (chip having a micrometer-order flow path) or a flow cell. The width of the flow path C is less than or equal to 1 mm, and in particular, may be greater than or equal to 10 μm but less than or equal to 1 mm. The flow path C and a flow path structure that includes this may be formed from a material such as plastic or glass.

The biological sample analysis apparatus according to the present disclosure is configured such that light from the light application unit 6101 is applied on a biological sample flowing inside the flow path C, in particular, on bioparticles within the biological sample. The biological sample analysis apparatus according to the present disclosure may be configured such that an irradiation point (interrogation point) for the light with respect to the biological sample is within the flow path structure formed by the flow path C, or may be configured such that an irradiation point for the light is outside of the flow path structure. As an example of the former, it is possible to give a configuration in which the light is applied on the flow path C within a microchip or a flow cell. For the latter, the light may be applied on bioparticles that have come out of the flow path structure (specifically, a nozzle thereof), and for example, it is possible to give a flow cytometer that uses a Jet in Air method.

### (Light Application Unit)

The light application unit 6101 includes a light source unit that emits light and a light guide optical system that guides the light to an irradiation point. The light source unit includes one or multiple light sources. The type of a light source is a laser light source or an LED, for example. The wavelength of light emitted from each light source may be any wavelength belonging to ultraviolet light, visible light, or infrared light. The light guide optical system includes, for example, an optical component such as a beam splitter group, a mirror group, or an optical fiber. In addition, the light guide optical system may include a lens group for condensing light, and includes an objective lens, for example. There may be one or multiple irradiation points at which the biological sample intersects with the light. The light application unit 6101 may be configured to condense light applied from one or multiple different light sources onto one irradiation point.

### (Detection Unit)

The detection unit 6102 includes at least one photodetector that detects light arising by light application onto bioparticles. Detected light is, for example, fluorescence or scattered light (any one or more of forward-scattered light, back-scattered light, and side-scattered light, for example). Each photodetector includes one or more light-receiving elements and, for example, has a light-receiving element array. As light-receiving elements, each photodetector may include one or multiple PMTs (photomultiplier tubes) and/or photodiodes such as APDs and MPPCs. For example, such a photodetector includes a PMT array in which multiple PMTs are arranged in one dimensional direction. In addition, the detection unit 6102 may include a CCD image capturing element, a CMOS image capturing element, etc. The detection unit 6102 can use this image capturing element to obtain an image (for example, a bright field-of-view image, a dark field-of-view image, a fluorescence image, etc.) of bioparticles.

The detection unit 6102 includes a detection optical system for causing light having a predetermined detection wavelength to reach a corresponding photodetector. The detection optical system includes a spectroscopic unit such as a prism or a diffraction grating or a wavelength separation unit such as a dichroic mirror or an optical filter. The detection optical system is configured to, for example, disperse light which has arisen by light application onto the bioparticles, such that the dispersed light is detected by multiple photodetectors that are greater in number than fluorochromes with which the bioparticles are labeled. A flow cytometer that includes such a detection optical system is referred to as a spectral flow cytometer. In addition, the detection optical system is configured to, for example, separate light corresponding to a fluorescence wavelength range for a specific fluorochrome from light that has arisen by light application onto bioparticles, and cause a corresponding photodetector to detect the separated light.

In addition, the detection unit 6102 can include a signal processing unit for converting an electrical signal obtained by a photodetector to a digital signal. This signal processing unit may include an A/D converter as a device for performing the conversion. A digital signal obtained by conversion using the signal processing unit can be transmitted to the information processing unit 6103. The digital signal can be handled as data pertaining to light (may also be referred to as “light data” hereinafter) by the information processing unit 6103. The light data may include fluorescence data, for example. More specifically, the light data may be light intensity data, and the light intensity may be light intensity data (may include feature amounts such as Area, Height, and Width) for light that includes fluorescence.

### (Information Processing Unit)

The information processing unit 6103, for example, includes a processing unit for executing processing on various items of data (for example, light data) and a storage unit for storing various items of data. In a case where light data corresponding to a fluorochrome is obtained by the detection unit 6102, the processing unit can perform a fluorescence leakage correction (a compensation process) on light intensity data. In addition, in a case of a spectral flow cytometer, the processing unit executes a fluorescence separation process on light data and obtains light intensity data corresponding to a fluorochrome. The fluorescence separation process may be performed in accordance with an unmixing method described in Japanese Patent Laid-open No. 2011-232259, for example. In a case where the detection unit 6102 includes an image capturing element, the processing unit may obtain morphological information for bioparticles according to an image obtained by the image capturing element. The storage unit may be configured to be able to store obtained light data. The storage unit may also be configured to be able to store spectral reference data used in the unmixing process.

In a case where the biological sample analysis apparatus 6100 includes the sorting unit 6104 which is described later, the information processing unit 6103 can, in reference to the light data and/or the morphological information, execute a determination on whether to sort bioparticles. Further, it is possible for the information processing unit 6103 to control the sorting unit 6104 according to a result of this determination, for sorting of the bioparticles to be performed by the sorting unit 6104.

The information processing unit 6103 may be configured to be able to output various items of data (for example, light data or an image). For example, the information processing unit 6103 can output various items of data (for example, a two-dimensional plot, a spectral plot, etc.) generated in reference to the light data. In addition, the information processing unit 6103 may be configured to be able to accept input of various items of data, and, for example, accepts from a user a gating process with respect to a plot. The information processing unit 6103 can include an output unit (for example, a display, etc.) or an input unit (for example, a keyboard, etc.), which are for causing execution of this output or input.

The information processing unit 6103 may be configured as a general-purpose computer, and, for example, may be configured as an information processing apparatus that includes a CPU, a RAM, and a ROM. The information processing unit 6103 may be included within a case in which the light application unit 6101 and the detection unit 6102 are provided, or may be provided outside of this case. In addition, various kinds of processing or functionality by the information processing unit 6103 may be realized by a server computer or a cloud connected via a network.

### (Sorting Unit)

The sorting unit 6104 executes sorting of bioparticles in response to a determination result by the information processing unit 6103. A method of sorting may be one using vibration to generate a droplet that includes bioparticles, applying a charge to the droplet which is a sorting target, and using an electrode to control a direction of travel for the droplet. The sorting method may be one performing sorting by controlling the direction of travel of bioparticles within a flow path structure. The flow path structure is provided with a control mechanism that uses pressure (injection or suction) or electric charge, for example. It is possible to give, as an example of the flow path structure, a chip (for example, a chip described in Japanese Patent Laid-open No. 2020-76736) that has a flow path structure in which a flow path C branches downstream into a recovery flow path and a waste liquid flow path, with specific bioparticles being recovered in the recovery flow path.

### (2) Example of Processing

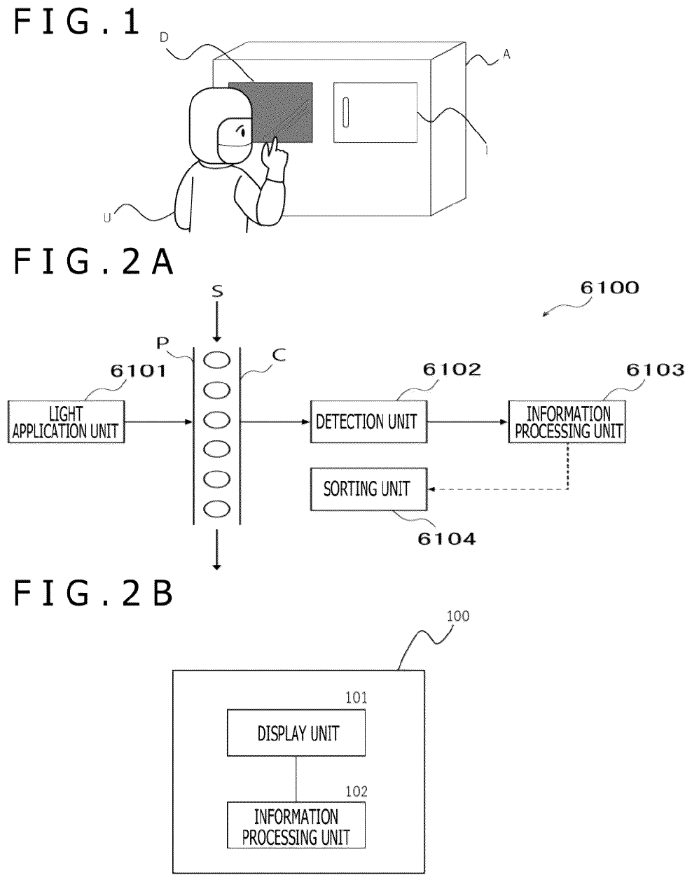

The bioparticle sorting apparatus according to the present disclosure may have a display unit D that is capable of touch input, as illustrated in FIG. 1, for example. As illustrated in the same figure, this display unit may be disposed on any surface, in particular, a side surface, of a housing for the apparatus. For example, a screen for the display unit may be disposed at a position where a standing (or sitting) user who is near the apparatus can perform touch input. A processing condition setting screen or processing execution operation screen that is described later may be displayed on this display unit. In addition, as illustrated in the same figure, the bioparticle sorting apparatus according to the present disclosure may have an introduction unit I for introducing a bioparticle-containing sample to the apparatus. For example, a container that includes this sample is inserted inside the apparatus from this introduction unit, and the container is connected to a predetermined sample line. Further, various kinds of processing described later may be executed after this connecting.

Note that, in the present specification, a touch operation may be a tap (single-tap), a long-tap, a double-tap, or the like, but there is no limitation to this. A touch operation may be a drag, a swipe, a pinch-in, or a pinch-out, for example. A touch operation type may be selected, as appropriate, by a person skilled in the art.

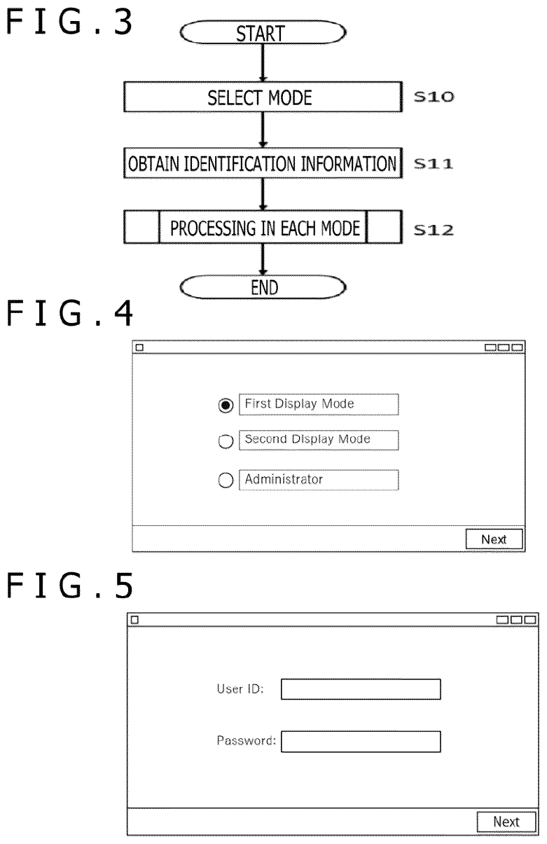

An example of processing executed by the bioparticle sorting apparatus according to the present disclosure is described below with reference to FIG. 2B and FIG. 3. FIG. 2B is an example of a block view of the bioparticle sorting apparatus. As illustrated in the same figure, a bioparticle sorting apparatus 100 according to the present disclosure has a display unit 101 and an information processing unit 102. FIG. 3 is an example of a flow chart for processing performed by this bioparticle sorting apparatus.

The display unit 101 outputs a screen according to the present disclosure. This screen may be based on data transmitted from the information processing unit 102. The display unit 101 may include a display apparatus known in the corresponding technical field. For example, the display unit 101 includes a display apparatus that has a rectangular screen, specifically, an oblong screen, and more specifically, a horizontally-long rectangular screen. It may be that the size of the screen is, for example, 10 inches or more, is desirably 11 inches or more, and is more desirably 12 inches or more. An upper limit for the size of the screen does not need to be set in particular, but, for example, may be 30 inches or less, 25 inches or less, 22 inches or less, or 20 inches or less. Such a screen size is suitable for display of images displayed in respective modes described below.

The information processing unit 102 causes the display unit 101 to display a screen, according to the present disclosure. The information processing unit 102 is configured to output data for display of this screen. The information processing unit 102 may be the information processing unit 6103 described in (1) above, but may be a separately prepared information processing unit. In addition, the bioparticle sorting apparatus 100 may include the light application unit 6101, the detection unit 6102, and the sorting unit 6104 described in (1) above, in addition to the display unit 101 and the information processing unit 102.

In the following (2-1) through (2-3), description is given regarding a screen that the bioparticle sorting apparatus 100 displays according to the present disclosure.

### (2-1) Selection of Display Mode

In step S10, the bioparticle sorting apparatus 100 (specifically, the information processing unit 102) starts processing.

In step S11, the information processing unit 102 causes the display unit 101 to output a mode selection screen for prompting a user to select a mode. As illustrated in FIG. 4, for example, the mode selection screen includes a button (First display mode button) for selecting the first display mode and a button (Second display mode button) for selecting the second display mode. This mode selection screen may also include a button (Next button) for causing the next processing screen to be displayed after the selection of the mode.

A more specific mode name may be displayed for each button displayed in the mode selection screen. For example, as the first display mode, a sorting process condition setting mode may be displayed or a setting mode, a Process Development mode, a PD mode, or the like may be displayed. In addition, as the second display mode, a processing execution operation mode may be displayed, or an operation mode, Operation mode, OP mode, or the like may be displayed.

As illustrated in the same figure, a button (Administrator button) for selecting an administrator mode may be additionally displayed in the mode selection screen. In the administrator mode, the information processing unit 102 can set or change privilege information for a user, for example.

In step S12, the information processing unit 102 causes the display unit 101 to display a screen for prompting input of identification information for a user. The identification information may include a user ID (or user name), and may additionally include a password. The screen has a user ID input field and a password input field as illustrated in FIG. 5, for example. The user inputs identification information to these fields.

The information processing unit 102 refers to inputted identification information to determine whether operation of the apparatus in the selected mode is permitted for the identification information. In a case of being permitted, the information processing unit 102 advances processing to step S13. In a case of not being permitted, the information processing unit 102, for example, causes the display unit 101 to display a screen indicating that permission is not given, and returns the processing to step S11 or S12.

In step S13, processing in the selected mode is executed. Description is given in (2-2) and (2-3) below regarding processing in each mode.

In the present disclosure, steps S12 and S13 may be omitted. In this case, the selection of one mode in step S11 may itself be used as identification information. Alternatively, it may be that only step S12 is executed instead of executing step S11. In other words, it may be that the information processing unit 102 causes the display unit 101 to display a screen for accepting input of identification information, and transitions to the first display mode or the second display mode according to the inputted identification information.

### (2-2) First Display Mode

In the first display mode, the bioparticle sorting apparatus 100 (specifically, the information processing unit 102) accepts input of operation control data pertaining to a bioparticle sorting process. The display unit 101 may be capable of touch input in order to accept input of this operation control data. The display unit 101 displays a processing condition setting screen for accepting input of operation control data pertaining to a sorting process for a bioparticle-containing sample. The operation control data may be data that is used to execute a sorting process in the second display mode, and in particular, includes data for generating a processing execution operation screen in the second display mode.

In the first display mode, the information processing unit 102 causes the display unit 101 to display a processing condition setting screen pertaining to the bioparticle sorting process. The information processing unit 102 accepts input of the operation control data via the processing condition setting screen.

Note that the processing condition setting screen may be displayed by another display apparatus instead of the display unit 101 which is included in the bioparticle sorting apparatus 100. For example, the bioparticle sorting apparatus 100 may cause the screen to be displayed by a display apparatus or an information processing apparatus that is connected, by wire or wirelessly, to the bioparticle sorting apparatus 100 via a network.

### (2-2-1) Example of Configuration of Screen Displayed in First Display Mode

### (Processing Condition Setting Screen)

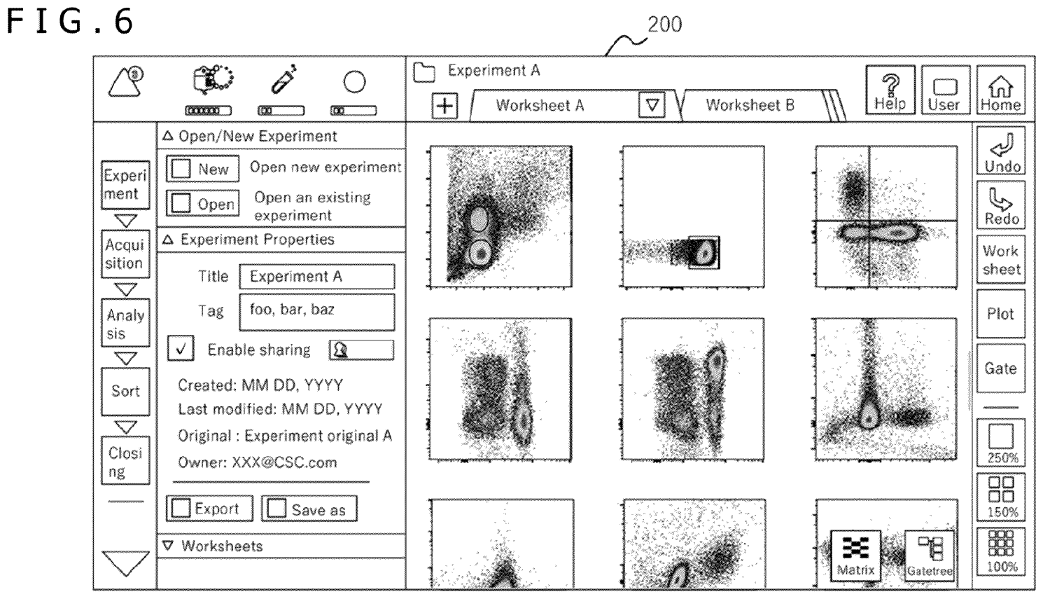

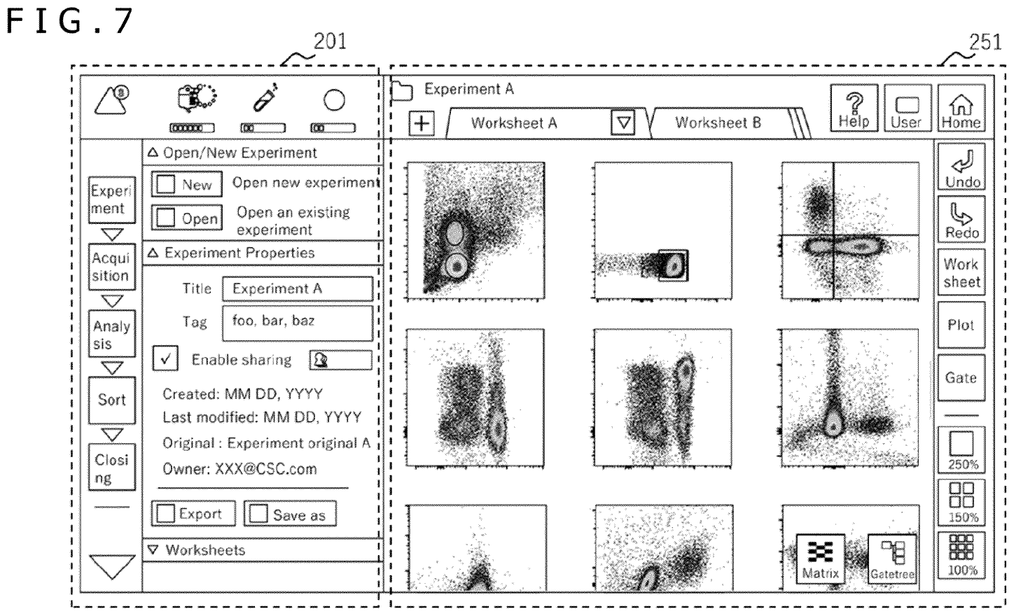

FIG. 6 illustrates an example of a configuration of the processing condition setting screen, which the bioparticle sorting apparatus 100 (specifically, the information processing unit 102) causes the display unit 101 to display. A processing condition setting screen 200 illustrated in the same figure includes, as illustrated in FIG. 7, a setting operation flow display area (hereinafter also referred to as a “first display area”) 201 for displaying an operation flow for setting a processing condition for a sorting process and a measurement data display area (hereinafter, “also referred to as a second display area”) 251 for displaying measurement data display area pertaining to a bioparticle-containing sample.

As illustrated in the same figure, it may be that the first display area is disposed on a left side in the processing condition setting screen, and the second display area is disposed on a right side in the processing condition setting screen.

Alternatively, it may be that the first display area is disposed on the right side in the processing condition setting screen, and the second display area is disposed on the left side in the processing condition setting screen.

In the first display mode, it is often the case that setting of detailed sorting conditions is required and, in conjunction with this, confirmation of detailed measurement data is necessary or display of many items of plot data on one screen is necessary. In contrast, presenting the first user with a work progress or operations that should be performed next is also required. Accordingly, by disposing the two areas on the left and right as described above, it is possible to ensure that the second display area is large. As a result, it is easier for the first user to confirm measurement data and the first user can confirm the work status through the display of the operation flow.

In order to improve operability, in a case where the first display area is disposed on the left side in the processing condition setting screen and the second display area is disposed on the right side in the processing condition setting screen, the first display area may be disposed to be in contact with the left end of the processing condition setting screen and the second display area may be disposed to be in contact with the right end of the processing condition setting screen.

Conversely, in a case where the first display area is disposed on the right side in the processing condition setting screen and the second display area is disposed on the left side in the processing condition setting screen, the first display area may be disposed to be in contact with the right end of the processing condition setting screen, and the second display area may be disposed so as to be in contact with the left end of the processing condition setting screen.

It may be that disposition of the first display area and the second display area can be changed. For example, the bioparticle sorting apparatus may be configured such that it is possible to change the screen from a state in which the former area is disposed on the right side and the latter area is disposed on the left side to a state in which the former area is disposed on the left side and the latter area is disposed on the right side. In addition, configuration may be taken such that it is possible to change the screen to achieve the opposite. As a result, it is possible to support convenience or handedness for a user, and it is possible to improve operability.

### (First Display Area)

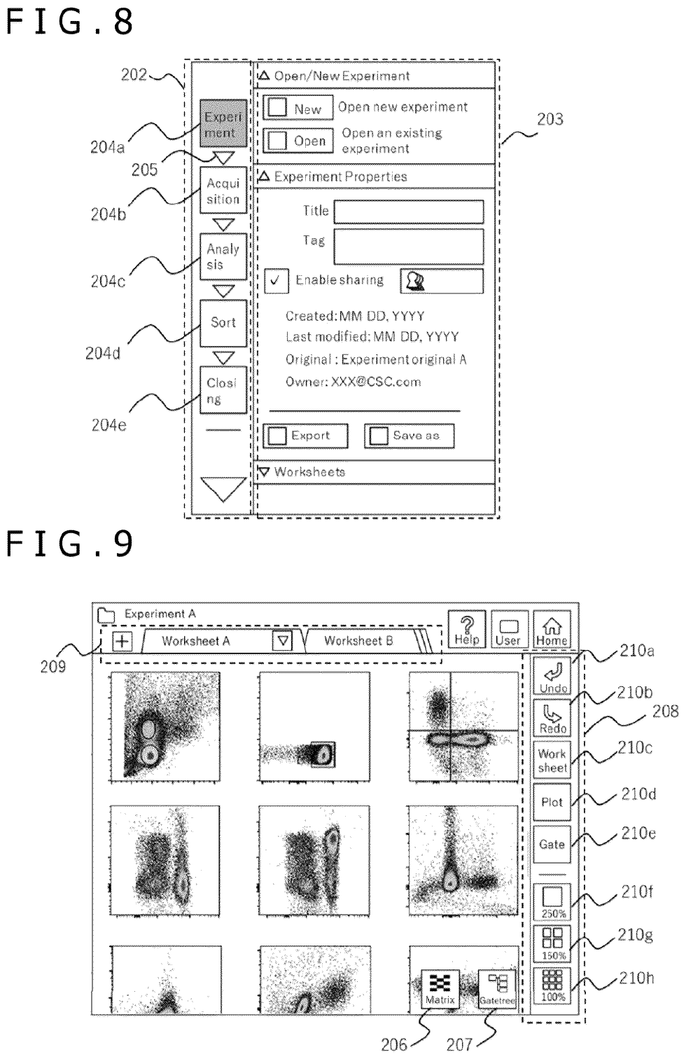

An example of the first display area is illustrated in FIG. 8. As illustrated in the same figure, the first display area includes a procedure button area 202 and a setting information display area 203.

Multiple processing buttons included in the operation flow are arranged, following an order for processing, in the procedure button area.

In the same figure, the first display area is disposed in a left-side end section of the processing condition setting screen, and the procedure button area is disposed in a left-side end section within the first display area. In addition, the procedure button area is disposed on the left side in the first display area, and the setting information display area is disposed on the right side in the first display area.

Alternatively, it may be that the first display area is disposed in a right-side end section of the processing condition setting screen, and the procedure button area is disposed in a right-side end section within the first display area. In addition, it may be that the procedure button area is disposed on the right side in the first display area, and the setting information display area is disposed on the left side in the first display area.

As a result, it is easy for the first user to find each button within a work flow. In addition, because the procedure button area is disposed at an end (specifically, a left end) of the processing condition setting screen, when touching a processing button, a possibility of touching another button decreases, and it is possible to have fewer erroneous operations due to touching an unintended portion.

It is often the case that a bioparticle sorting apparatus user interface in the related art is presupposed on being operated via a display for a desktop or laptop personal computer. In this case, for example, a cursor is operated by a mouse or the like, and a tool button having a high frequency of being selected is disposed at the top of the screen as in a typical software window.

However, in a case where a user operates a touch panel as illustrated in FIG. 1 described above, when tool buttons having a high frequency of being selected are disposed at the top of a screen, these tool buttons are difficult to touch. In addition, in a case of causing a finger to move toward these tool buttons, the possibility of touching another button also increases.

By, according to the present disclosure, the procedure button area being disposed on the left side (or right side) of the screen, in particular, being disposed at the left end (or right end), access to buttons within the procedure button area becomes easier, and the possibility of touching another button is also low. Accordingly, it is possible to provide a screen that is easy to use and easy to operate for the first user.

The same figure is an example of the first display area in a case where the first display area is disposed on the left side in the processing condition setting screen and the second display area is disposed on the right side in the processing condition setting screen.

Note that, in a case where the first display area is disposed on the right side in the processing condition setting screen and the second display area is disposed on the left side in the processing condition setting screen, the first display area may be disposed to be in contact with the right end of the processing condition setting screen, and the procedure button area may be disposed on the right side in the first display area and disposed at the right end of the processing condition setting screen in particular.

The procedure button area 202 includes multiple processing buttons 204a through 204e. The buttons respectively indicate multiple steps performed from the start to the completion of the creation of operation control data. The multiple processing buttons may be aligned from the top to the bottom of the screen as illustrated in the same figure. By the multiple processing buttons being aligned in one direction in such a manner, it is possible to understand the order of each step, and it is easier to understand work progress. A mark 205 (for example, a downward-facing triangle illustrated in the same figure, an arrow, or the like) for indicating that there is an order for these steps may be disposed between two processing buttons.

Among the multiple processing buttons, the color or shape of a button that indicates a step for which work is underway may be made to be different from the color or shape of other buttons. For example, as illustrated in the same figure, it may be that the processing button 204a indicating a step for which work is underway is indicated by gray, and the processing buttons 204b through 204e indicating other steps are indicated by white. As a result, it becomes easier to identify a step for which work is underway.

The shape of the multiple processing buttons may be a polygon (for example, a rectangle) or a round shape. The size and disposition interval for the multiple buttons may be set such that two or more buttons are not selected in a case where a touch is performed by a fingertip, for example. In a case where each button is a rectangle, it may be that the long sides (or sides) are, for example, greater than or equal to 0.3 cm, and desirably, greater than or equal to 0.5 cm. In addition, it may be that the long sides (or sides) are, for example, less than or equal to 5 cm, and desirably, less than or equal to 3 cm. In a case where each button is a round shape, it may be that a diameter (or long diameter) is, for example, greater than or equal to 0.3 cm, and desirably, greater than or equal to 0.5 cm. In addition, it may be that the diameter (or long diameter) is, for example, less than or equal to 5 cm, and desirably, less than or equal to 3 cm.

Content corresponding to a step selected by a respective procedure button is displayed in the setting information display area 203. More specifically, in response to any processing button being touched, the setting information display area 203 displays setting information corresponding to the processing button. Description is given below regarding an example of this displayed content.

### (Second Display Area)

As illustrated in FIG. 9, measurement data is displayed in the second display area 251. The second display area may be configured to be able to display one or more plots and, for example, may include multiple items of plot data as illustrated in the same figure. The multiple items of plot data may be disposed in a grid shape. In addition, a display format for the multiple items of plot data may be changed to a list shape (for example, to line up from above to below). It is possible to set a gate for each item of plot data. In other words, the second display area may be configured to accept, according to a touch operation, input of gate information for identifying bioparticles to be sorted in the process.

The second display area may display one or more operation buttons, or may display one or more operation buttons for displaying data associated with the one or more plots.

The associated data is fluorescence correction matrix data, for example. A matrix display button 206 is illustrated in the same figure as an example of an operation button for displaying the fluorescence correction matrix data. This matrix display button (Matrix button) is for displaying a window for displaying and/or adjusting a fluorescence correction matrix used to generated plot data that is displayed in the measurement data area.

In addition, the associated data may be back data used to generate the plot, for example. The back data may include statistic information for each gate, for example. A gate tree display button 207 is illustrated in the same figure as an example of an operation button for displaying the statistic information. This gate tree display button (Gatetree button) is for displaying a window for displaying a gate tree for plot data displayed in the measurement data area.

As illustrated in the same figure, these operation buttons may be disposed within the measurement data display area, and in particular, may be disposed in a right-side portion (specifically, a bottom-right portion) in the second display area. As a result, access is easy for a user's finger.

Note that in a case where the second display area is disposed on the left side of a screen, these operation buttons may be disposed in a left-side portion (specifically, a bottom-left portion) of the second display area. In addition, these buttons may be disposed within an operation button area described below.

Description is given below regarding details of a window displayed by these operation buttons being touched.

The second display area may further include an operation button area 208. In addition, the second display area may further include a tab area 209.

The operation button area is disposed on the right side of the second display area and, in particular, is disposed at a right end of the second display area. As a result, it is easy for the first user to find each operation button.

In addition, because the operation button area is disposed at an end (right end) of the processing condition setting screen 200, a possibility of touching another button when touching an operation button decreases, and it is possible to have fewer erroneous operations due to touching an unintended portion.

However, in a case where a user operates a touch panel as described above, when tool buttons having a high frequency of being selected are disposed at the top of a screen, these tool buttons are difficult to touch. In addition, in a case of causing a finger to move toward these tool buttons, the possibility of touching another button also increases.

By, according to the present disclosure, the operation button area being disposed on the right side (or left side) of the screen, in particular, being disposed at the right end (or left end), access to buttons within the operation button area becomes easier, and the possibility of touching another button is also low. Accordingly, it is possible to provide a screen that is easy to use and easy to operate for the first user.

The same figure is an example of the second display area in a case where the first display area is disposed on the left side in the processing condition setting screen, and the second display area is disposed on the right side in the processing condition setting screen.

Note that, in a case where the first display area is disposed on the right side in the processing condition setting screen and the second display area is disposed on the left side in the processing condition setting screen, the second display area may be disposed to be in contact with the left end of the processing condition setting screen, and the operation button area may be disposed on the left side in the setting second display area and disposed at the left end of the processing condition setting screen in particular.

Multiple operation buttons 210a through 210h are displayed in the operation button area 208. Each operation button is a button for performing an operation on measurement data displayed on a screen. As illustrated in the same figure, the multiple operation buttons may be lined up from top to bottom in one column in the screen, but may be lined up in two or more columns, for example.

Note that, in the same figure, the operation buttons 210a through 210h are disposed within the operation button area, but one or more of these operation buttons may be disposed within the measurement data display area.

The shape of the multiple operation buttons may be a polygon (for example, a rectangle) or a round shape. The size and disposition interval for the multiple buttons may be set such that two or more buttons are not selected in a case where a touch is performed by a fingertip, for example. In a case where each button is a rectangle (an oblong or a square), it may be that the long sides (or sides) are, for example, greater than or equal to 0.3 cm, and desirably, greater than or equal to 0.5 cm. In addition, it may be that the long sides (or sides) are, for example, less than or equal to 5 cm, and desirably, less than or equal to 3 cm. In a case where each button is a round shape, it may be that a diameter (or long diameter) is, for example, greater than or equal to 0.3 cm, and desirably, greater than or equal to 0.5 cm. In addition, it may be that the diameter (or long diameter) is, for example, less than or equal to 5 cm, and desirably, less than or equal to 3 cm.

The multiple operation buttons may include an Undo button 210a and a Redo button 210b, for example. The former is a button for canceling an operation with respect to data on a screen. The latter is a button for re-doing an operation with respect to data on a screen, or re-executing a canceled operation.

The multiple operation buttons may include one or more toolbox display buttons. As examples of toolbox display buttons, it is possible to give a worksheet toolbox display button 210c (Worksheet button), a plot toolbox display button 210d (Plot button), and a gate toolbox display button 210e (Gate button). The multiple operation buttons may include one, two, or all three of these.

The worksheet toolbox button is for displaying a toolbox that includes multiple buttons for controlling a display format for plot data displayed in the measurement data area.

The plot toolbox display button is for displaying a toolbox that includes multiple buttons for adjusting content for each item of plot data.

The gate toolbox display button is for displaying a toolbox that includes multiple buttons for adjusting gate information set for each item of plot data.

Description is given below regarding details of these toolboxes.

The multiple operation buttons may further include multiple buttons for changing a display magnification for plot data displayed in the second display area. For example, as illustrated in FIG. 9, the multiple operation buttons may include the multiple buttons 210f through 210h for displaying respective item of plot data at a predetermined magnification. In addition, the multiple operation buttons may include a button for increasing the magnification and/or a button for decreasing the magnification.

The tab area 209 is an area in which are displayed one or more tabs for displaying, in the second display area, only any one of multiple open worksheets. A certain tab is selected, whereby the tab becomes active, and a worksheet associated with the tab is displayed within the second display area. A worksheet associated with an inactive tab is not displayed. In FIG. 9, a tab for a worksheet A is active, and a plot data group included in the worksheet A is being displayed. In the same figure, a worksheet B is also included in the same experiment file, but the worksheet B is inactive, and a plot data group included in the worksheet B is not being displayed.

In the present specification, a worksheet associated with an active tab (also referred to as an active worksheet) may mean a worksheet that is a target of a sorting process. A bioparticle sorting apparatus may execute a sorting process for bioparticles according to gate information for the active worksheet.

In addition, an inactive tab is a tab associated with another worksheet apart from the active worksheet. A worksheet associated with an inactive tab may be referred to as an inactive worksheet. An inactive worksheet may be, for example, a worksheet for a sorting process executed in the past, and/or a reference worksheet, or the like. It may be that inactive tabs are displayed after being grouped at an end (for example, the right end or the left end) of the tab area 209.

A display position for an active tab in the tab area is desirably immobilized. For example, the tab area may be displayed such that a tab selected by the first user is present at the left end (or right end) of the tab area. In this case, an inactive tab group is disposed at the right end (or left end).

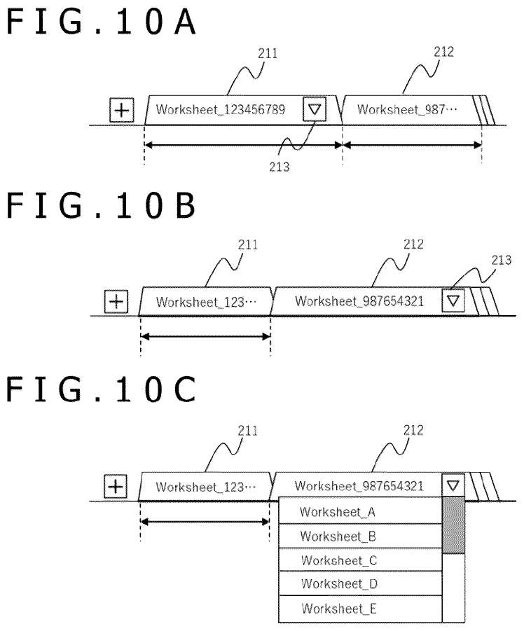

The size of the tab may be constant, but is desirably changed dynamically. For example, the information processing unit 102 causes the size in the horizontal direction (arrangement direction for text) for a selected tab to be displayed larger than the size in the horizontal direction for an unselected tab. As a result, it is possible to present a user with a selected tab with emphasis. As illustrated in FIG. 10A, for example, display is performed such that the size in the horizontal direction for an active tab 211 is greater than the size of an inactive tab 212.

The size of a tab may be changed in conjunction with the position of the tab being changed or may be changed according to whether or not the tab is selected, irrespective of a change of the position of the tab.

In addition, the information processing unit 102 may change the size of a tab such that the entirety of a worksheet name for an active tab is displayed within the tab. As a result, it is possible to present a user with a selected tab with emphasis. For example, in FIG. 10A, for the active tab 211, the entirety of the file name thereof, “Worksheet_123456789,” is displayed. In contrast, in FIG. 10B, the tab 211 is inactive, and in conjunction with this, a portion of the file name, “Worksheet_123,” and an ellipsis, “ . . . ,” are displayed within the tab 211.

The information processing unit 102 can, for example, cause one tab that is active and one or two other tabs to be displayed in the tab area together with at least a portion of the worksheet name thereof. One or more tabs other than these may be configured to be displayed by being pulled-down and, for example, may be configured to be displayed in a pull-down menu that is displayed by a predetermined button being touched or selected. As a result, it is possible to intuitively understand which is the active worksheet (specifically, a worksheet for which measurement is underway). In addition, even in a case where many worksheets are open on a narrow screen, it is possible to easily confirm a worksheet name for which there is a desire to make a comparison with or refer to, and it is also possible to switch worksheets by one touch or click.

For example, a pull-down button such as that indicated by a reference symbol 213 in FIGS. 10A and 10B may be displayed within the tab area. It may be that, by this pull-down button being selected, a pull-down menu is displayed as illustrated in FIG. 100, for example. Worksheets not displayed in the tab area may be listed within the pull-down menu. Note that the pull-down button is disposed within the active tab in FIGS. 10A and 10B, but may be disposed within an inactive tab or may be disposed at another position within the tab area. In such a manner, the information processing unit 102 may cause the display in the tab area of a pull-down button that, according to being pulled-down, displays a list of worksheets that are not displayed.

### (2-2-2) Example of Processing in First Display Mode

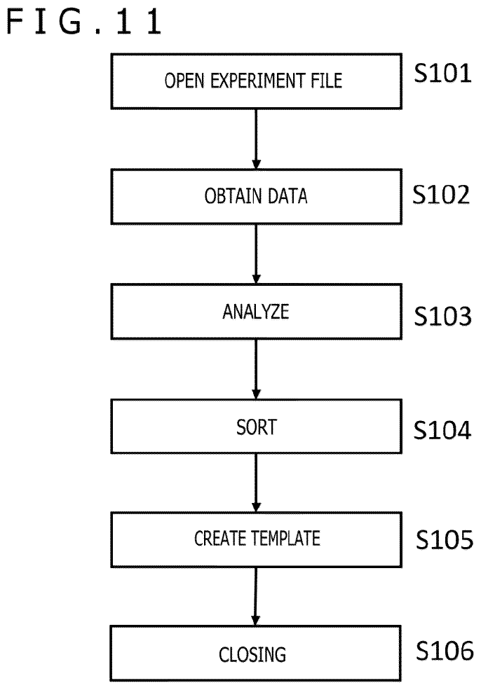

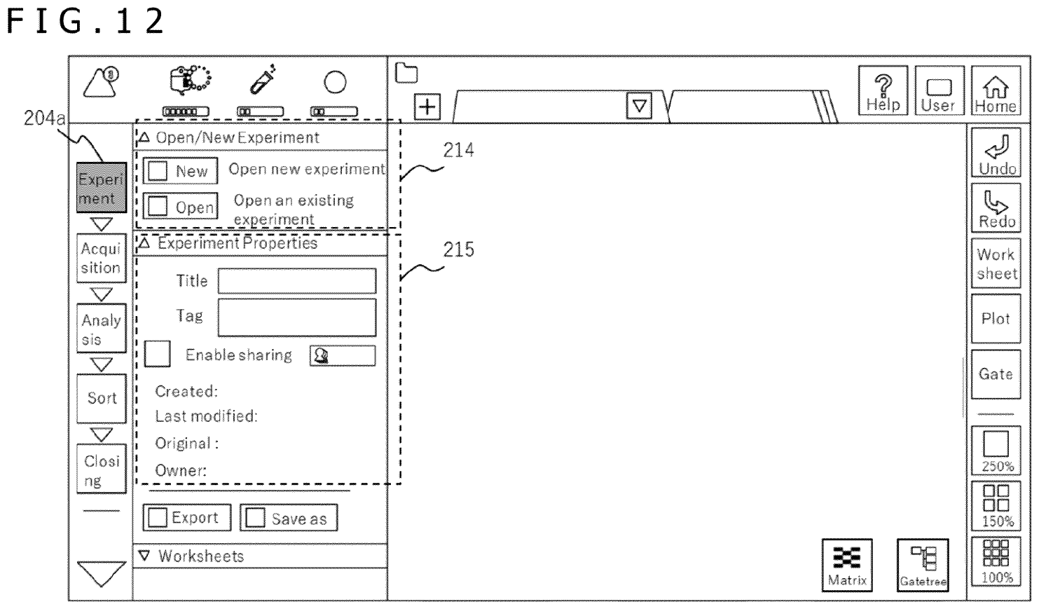

FIG. 11 illustrates an example of a flow chart for a case in which processing in the first display mode is performed in step S13 described in (2-1) above. With reference to the same figure, description is given regarding processing executed by the bioparticle sorting apparatus in the first display mode, in particular, regarding an example of a screen displayed in this processing.

### (File Open Step S101)

When processing in step S13 is started, the information processing unit 102 causes the display unit 101 to display a processing condition setting screen illustrated in FIG. 12. At a time of starting this processing, as illustrated in the same figure, a processing button (indicated as an Experiment button in the same figure) 204a for newly creating an experiment file or opening an existing experiment file may be selected in the first display area (specifically, the procedure button area).

In a state where the Experiment button is selected, the information processing unit 102 causes a file-open area 214 (Open/New Experiment area), in which a button for newly creating an experiment file and a button for opening an existing experiment file are displayed, and a property area 215 (Experiment properties area) that displays properties of an experiment file, to be displayed in the setting information display area, as illustrated in the same figure.

In response to the first user selecting the button (indicated as a New button in the same figure) for newly creating an experiment file in the file-open area, the information processing unit 102 blanks a title box (box to which Title has been added) and a tag box (box to which Tag has been added) in the property area, and also blanks the second display area. The title box is a field to which name data for an experiment file is inputted. The tag box is a field to which a tag (keyword) for searching for an experiment file is inputted.

The first user inputs title data and tag data in the property area. The inputted title data and tag data may be included in the operation control data.

In response to the first user selecting the button (indicated as the Open button in the same figure) for opening an existing experiment file, the information processing unit 102 displays a screen that allows the first user to select an existing experiment file. In response to the first user selecting an existing experiment file in this screen, the information processing unit 102 respectively outputs title data and tag data included in this existing experiment file to the title box and the tag box.

In the processing condition setting screen illustrated in FIG. 6, for example, “Experiment A” is outputted as the experiment file name, and “foo, bar, baz” are outputted as tags.

The property area may display other property data pertaining to operation control data, as illustrated in the same figure. For example, the other property data can include, inter alia, a creation date and a final update date for the operation control data. The other property data may also be included in the operation control data.

As illustrated in the same figure, the setting information display area may also display an export button (indicated as an Export button in the same figure) for exporting open operation control data and/or a save button (indicated as a Save button in the same figure) for saving the operation control data.

The first user selects an Acquisition button from within the procedure button area in order to obtain data pertaining to a bioparticle-containing sample, for example, after having inputted property data into the property area in the case of having created a new experiment file or after having confirmed property data within the property area in a case where an existing experiment file is open.

### (Data Obtainment Step S102)

In response to the Acquisition button being selected, the information processing unit 102 changes the color of the button in the first display area (in particular, the procedure button area) to indicate a state in which the Acquisition button is selected.

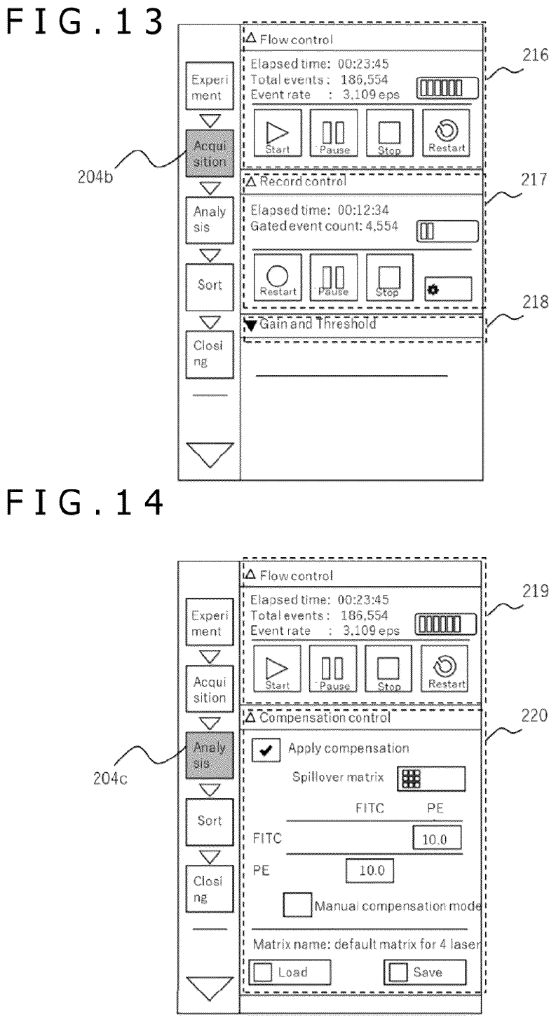

In response to the Acquisition button being selected, the information processing unit 102 causes the setting information display area to display a screen as illustrated in FIG. 13. This screen is for controlling processing for obtaining data (light data, in particular, scattered light data and/or fluorescence data) pertaining to bioparticles in the bioparticle-containing sample. In this state, the information processing unit 102 causes the setting information display area to display a flow control area 216 (Flow control area) and a recording control area 217 (Record control area), as illustrated in the same figure.

As illustrated in the same figure, a button group, which is for controlling an operation for supplying the bioparticle-containing sample to the flow path on which the sorting process is to be performed, and data indicating a status of the operation are displayed in the flow control area. As illustrated in the same figure, this button group includes a start button (Start button) that starts the operation for supplying the sample (or restarting a paused operation), a pause button (Pause button) for pausing the operation, an end button (Stop button) for ending the operation, and a restart button (Restart button) for re-executing an operation that has ended. In addition, an elapsed amount of time for the operation, the number of events detected for the operation, a detection ratio (the number of detected events per an amount of time), or the like may be displayed in the flow control area.

As illustrated in the same figure, the recording control area displays a button group for controlling recording of a sorting determination result based on a gate that has been set as well as data indicating a status of the recording. As illustrated in the same figure, this button group includes a start button (Record button) for starting the recording (or re-starting paused recording), a pause button (Pause button) for pausing the recording, and an end button (Stop button) for ending the recording. In addition, the recording control area may display an elapsed amount of time for the recording operation, the number of events that have become analysis targets, etc.

In addition, in a state where the Acquisition button is selected, the bioparticle sorting apparatus 100 may cause the setting information display area to display an area 218 for setting a gain (gain) and/or a threshold (threshold). The bioparticle sorting apparatus 100 may be configured to enable a gain and/or a threshold for a detection unit to be set in this area. Using these settings, it is possible to control a type or range of events that are analysis targets. Note that this area may be closed in the same figure. In response to a user selecting this area, an area for performing this setting is deployed.

The first user inputs a setting pertaining to a gain and/or a threshold in this area. Setting data pertaining to the inputted gain and/or threshold may be included in a portion of the operation control data and, in particular, may be handled as a portion of the abovementioned “(a) processing condition data employed by a processing apparatus that executes the processing in the second mode.”

The first user selects an Analysis button 204c from within the procedure button area, after light detection data having a predetermined number of events is obtained. Alternatively, the information processing unit 102 may automatically transition to a state in which the Analysis button 204c is selected if data having a preset number of events is obtained.

### (Analysis Step S103)

In response to the Analysis button being selected, the information processing unit 102 changes the color of the button in the first display area (in particular, the procedure button area) to indicate a state in which the Analysis button is selected.

In response to the Analysis button being selected, the information processing unit 102 causes the setting information display area to display a screen as illustrated in FIG. 14. This screen is for setting a gate for sorting intended bioparticles in reference to the light data obtained in the data obtainment step. In a state where the Analysis button is selected, the bioparticle sorting apparatus 100 causes the setting information display area to display a flow control area 219 and a fluorescence correction setting area 220 (displayed as a Compensation control area in the same figure).

The flow control area 219 is the same as the flow control area 216 described in relation to the Acquisition step above, and a button group which is for controlling an operation for supplying the bioparticle-containing sample to the flow path on which the sorting process is to be performed and data indicating a status of the operation are displayed in the flow control area.

As illustrated in the same figure, the fluorescence correction setting area 220 displays a checkbox for selecting whether to apply a fluorescence correction (displayed as Apply compensation in the same figure) and one or more correction coefficient input boxes (the number 10.0 is inputted in the same figure) for adjusting a correction coefficient included in a correction matrix that has been read in. The correction matrix is adjusted using input of numerical values to the correction alphanumeric input box.

In addition, this area may display the name of the correction matrix to be applied (displayed as default matrix for 4 laser in the same figure), a button for reading in the correction matrix (Load button), and a button (Save button) for, in a case where a correction matrix that has been read in is adjusted, saving the adjusted correction matrix.

The first user inputs a setting pertaining to the correction matrix (such as a correction coefficient) in this area. Setting data pertaining to an inputted correction matrix may be included in a portion of the operation control data and, in particular, may be included as a portion of the abovementioned “(a) processing condition data employed by a processing apparatus that executes the processing in the second mode.”

In a state where the Analysis button is selected, the first user inputs gate information by performing a gate setting in the second display area. Inputted gate information may be included in a portion of the operation control data and, in particular, may be included as a portion of the abovementioned “(a) processing condition data employed by a processing apparatus that executes the processing in the second mode.”

Setting of the gate information may be executed, as appropriate, by the first user. For example, plot data is generated from obtained light data, a gate is set for the generated plot data, plot data is further generated after deploying the gate, and a gate is further set with respect to this plot data. By repeating, as appropriate, operations such as plot data generation, gate setting, and deployment in such a manner, gate information for sorting intended bioparticles is generated. Generated plot data may be a two-dimensional plot or a one-dimensional plot, and the type of plot data may be selected, as appropriate, by the first user. In addition, the first user may select, as appropriate, scattered light/a fluorescence channel which is employed as axes in each item of plot data as well as scales for the axes.

The information processing unit 102 causes the second display area to display one or more items of plot data generated in order to set the gate information described above. The generated one or more items of plot data may be included in the operation control data. For example, any one or more items of image data among the generated one or more items of plot data may be included in the operation control data, specifically, may be included as a portion of the “operator-directed instruction data that is directed at an operator who operates the processing apparatus in the second mode” in (c) above, and more specifically, may be included as data to be referred to by the second user.

In generation of the gate information, a window displayed by the matrix display button and/or the gate tree display button being selected may be referred to. Description is given below regarding these.

### (Matrix Display Button)

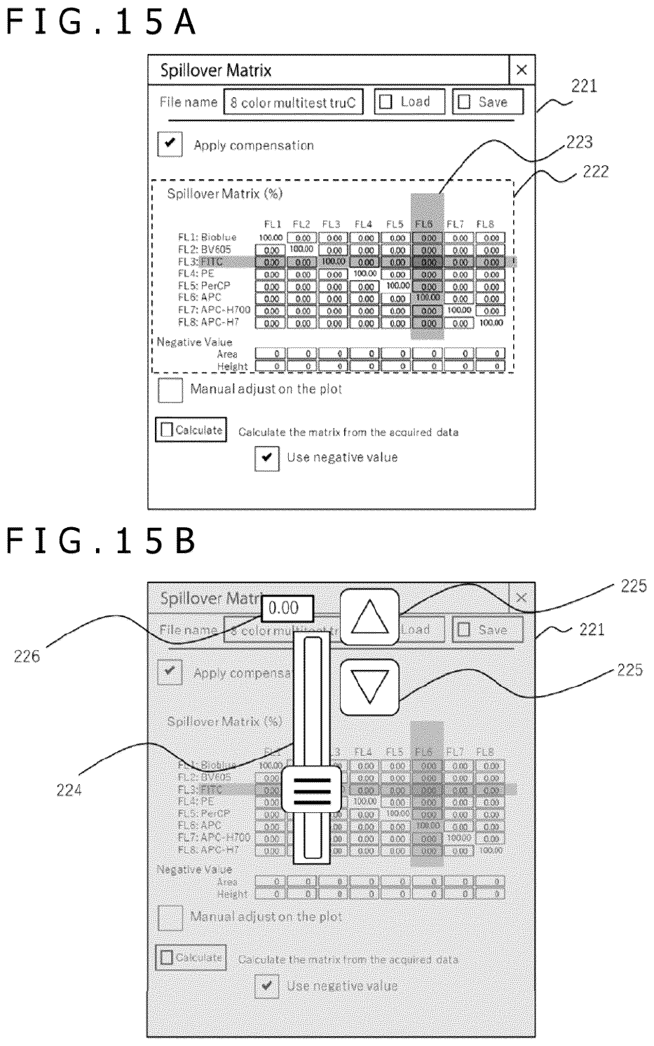

FIG. 15A illustrates a window displayed by the matrix display button being displayed. As illustrated in the same figure, a window 221 includes a correction matrix 222 that is being applied to displayed measurement data. The size of the window may be smaller than the size of the second display area. For example, the surface area of the window may be, for example, less than or equal to 60% of the surface area of the second display area, and desirably, less than or equal to 50%. In addition, the surface area of the window may be, for example, greater than or equal to 20% of the surface area of the second display area, and desirably greater than or equal to 30%. As a result, it is possible to adjust a parameter in the correction matrix while checking measurement data being displayed in the second display area.

In addition, as illustrated in the same figure, the window may include a file name display field (to which File name is added in the same figure) for displaying a file name for the correction matrix, a read-in button (Load button) for reading in the correction matrix, and a save button (Save button) for saving an adjusted correction matrix.

As illustrated in the same figure, the window may also include a checkbox for selecting whether to apply a correction, a checkbox for selecting whether to make an adjustment by a manual operation on a plot, a Calculate button for calculating a matrix from obtained data, and a checkbox for selecting whether to use a Negative Value.

It may be that, in response to the first user selecting one correction coefficient from the correction matrix, the information processing apparatus 102 may perform image processing to emphasize the selected correction coefficient, such as by displaying a cross-shaped indicator 223 for emphasizing the selected correction coefficient. As a result, it is easier for the first user to confirm the correction coefficient that the first user selected him/herself.

The information processing unit 102 may be configured to accept adjustment of each correction coefficient in the correction matrix. A status of the adjustment is illustrated in FIG. 15B. As illustrated in the same figure, according to a finger belonging to the first user selecting a correction coefficient for which there is a desire to adjust, the information processing apparatus 102 causes a slider 224 to be displayed near the selected position. In conjunction with this, the information processing apparatus 102 may execute image processing for emphasizing the slider, such as by adding a gray color to the window 221 as illustrated in the same figure.

As illustrated in the same figure, the slider may be an indicator that can move in an up-down direction or may be an indicator that can move in a left-right direction or any other two directions.

The information processing unit 102 may cause a slider movement button 225 for moving the slider to be displayed near the slider. The slider may be moved in response to the button being selected.

The information processing unit 102 may cause a box 226, which is for displaying the selected correction coefficient, to be enlarged and displayed near the slider. As a result, it is easy for the first user to confirm the correction coefficient value that the first user selected him/herself. In response to movement by the slider or the button being touched, the information processing apparatus 102 changes the correction coefficient within the box.

The first user may use an operation with respect to the window to adjust the correction matrix. In response to the first user selecting the save button after the adjustment, the information processing apparatus 102 saves the adjusted correction matrix data. The adjusted correction matrix data may be included in the operation control data and, in particular, may be included as a portion of the abovementioned “(a) processing condition data employed by a processing apparatus that executes the processing in the second mode.”

Desirably, display or hiding of the window may be switched according to the matrix display button being touched and, in other words, the matrix display button may be a toggle button. In response to the matrix display button being touched by a user, the information processing unit 102 causes the window to be displayed within the processing condition setting screen, and in response to the matrix display button being touched by the user again thereafter, the information processing unit 102 closes the displayed window.

Although it is envisioned that the window will be frequently checked by the first user, it will be more difficult to check plot data within the measurement data display area when the window is always displayed. Accordingly, the matrix display button is made to be a toggle button as described above, whereby it is possible to improve the convenience of the window while making it easier to check plot data.

The matrix display button is desirably disposed at an end of the processing condition setting screen, particularly, an end of the second display area. As a result, it becomes easier for a user to touch the matrix display button. In FIG. 9, the matrix display button 206 is disposed within the measurement data display area in the second display area, but this button may be disposed within the operation button area.

### (Gate Tree Display Button)

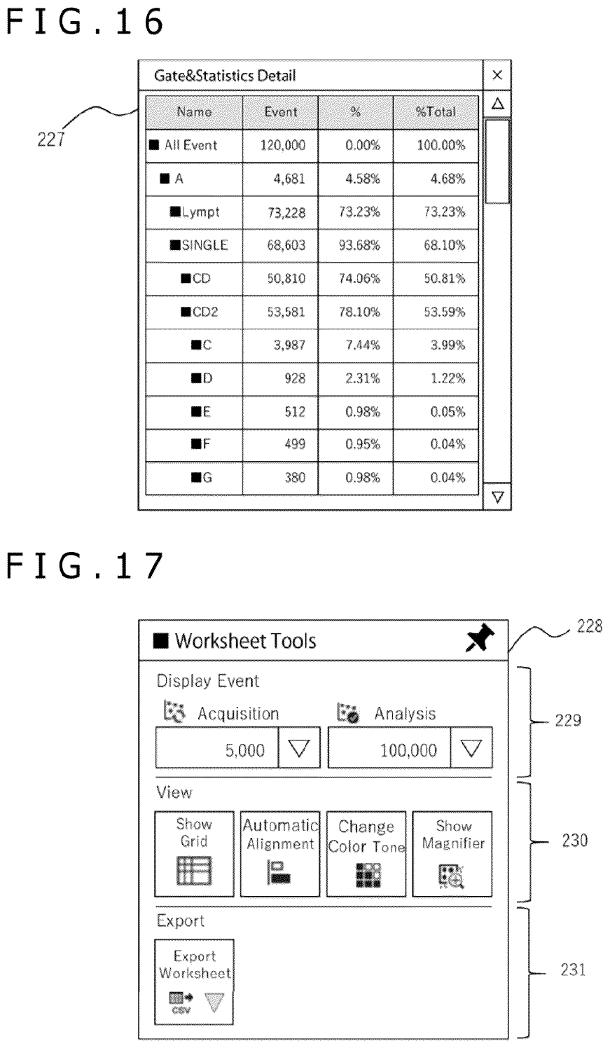

FIG. 16 illustrates an example of a window displayed by the gate tree display button being selected. A window 227 illustrated in the same figure indicates statistical data for a displayed measurement data group. The size of the window may be smaller than the size of the second display area. For example, the surface area of the window may be, for example, less than or equal to 60% of the surface area of the second display area, and desirably, less than or equal to 50%. Further, the surface area of the window may be, for example, greater than or equal to 20% of the surface area of the second display area, and desirably, greater than or equal to 30%. As a result, it is possible to adjust a parameter in the correction matrix while checking the measurement data being displayed in the second display area.

The statistical data includes a name for each gate. Moreover, the statistical data may include one, two, or all three of the number of events included in each gate, a ratio of the number of events included in each gate among the number of events displayed in certain plot data, and a ratio of the number of events included in each gate with respect to the total number of events. The statistical data indicated in the same figure includes the number of events corresponding to each gate and the abovementioned two types of ratios, together with the name of each gate.

The information processing unit 102 may be configured to change the statistical data in response to respective gates being adjusted. The statistical data is frequently checked in a bioparticle sorting process. Hence, by the statistical data being changed in conjunction with adjustment of a gate, the first user can efficiently check the impact of adjusting the gate.

Desirably, display or hiding of the window may be switched according to the gate tree display button being touched, and in other words, the gate tree display button may be a toggle button. In response to the gate tree display button being touched by a user, the information processing unit 102 causes the window to be displayed within the processing condition setting screen, and in response to the gate tree display button being touched by the user again thereafter, the information processing unit 102 closes the displayed window.

Although it is envisioned that this window will also be frequently checked by the first user, it will be more difficult to check plot data within the measurement data display area when the window is always displayed. Accordingly, the gate tree display button is made to be a toggle button as described above, whereby it is possible to improve the convenience of the window while making it easier to check plot data.

The gate tree display button is desirably disposed at an end of the processing condition setting screen, particularly an end of the second display area. As a result, it becomes easier for a user to touch the gate tree display button. In FIG. 9, the gate tree display button 207 is disposed within the measurement data display area in the second display area, but this button may be disposed within the operation button area.

In order to generate the gate information, it is possible to use a toolbox group displayed by the worksheet toolbox display button, the plot toolbox display button, and the gate toolbox display button being selected. Description is given below regarding these.

### (Worksheet Toolbox)

FIG. 17 illustrates an example of a toolbox displayed by the worksheet toolbox display button being selected. A toolbox 228 indicated in the same figure includes a button group for adjusting a setting applied to one entire worksheet or for performing processing on one entire worksheet.

For example, as illustrated in the same figure, this toolbox may include one, two, or three from among an area 229 that includes one or more buttons for adjusting the number of events which are to be display targets for a worksheet, an area 230 that includes one or more buttons for adjusting a display format for plot data in the worksheet, and an area 231 that includes one or more buttons for exporting the worksheet.

The first user uses this toolbox to perform a setting for the worksheet. As a result, worksheet setting data is generated. In other words, the worksheet setting data includes any one or more items of data inputted via the toolbox. Data inputted in such a manner may be included as a portion of the operation control data, or included as a portion of the “(b) display control data pertaining to display for the processing execution operation screen in the second mode,” for example. For example, data regarding the number of events that specifies the number of events to be display targets (data inputted via the area 229), display format data (data inputted via the area 230) that specifies a display format for plot data, or both of these may be included in the display control data in the above (b). Note that these items of data may be handled as one that is included in the “(a) processing condition data employed by a processing apparatus that executes the processing in the second mode.”

### (Plot Toolbox)

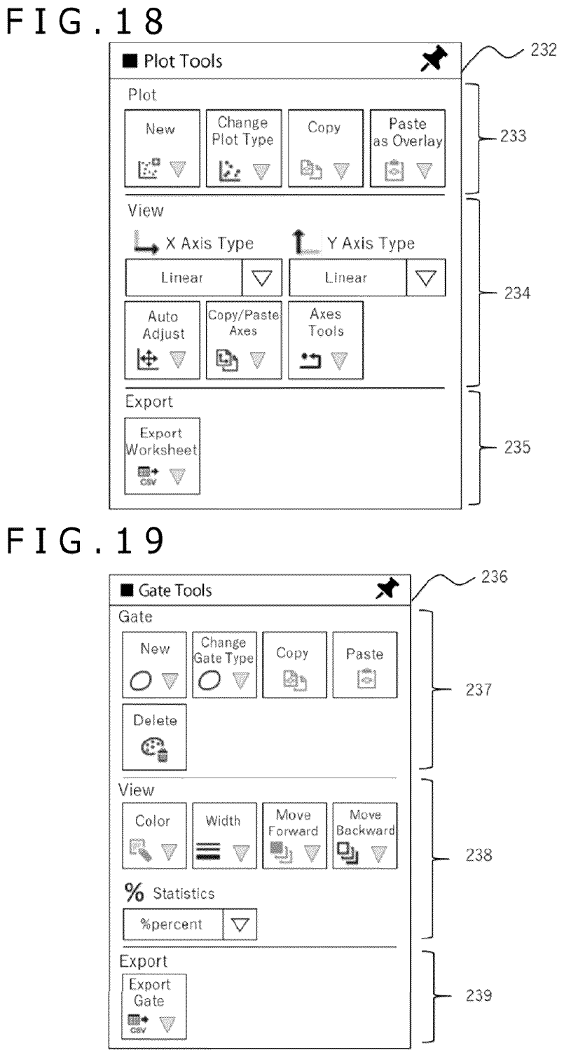

FIG. 18 illustrates an example of a toolbox displayed by the plot toolbox display button being selected. A toolbox 232 indicated in the same figure includes a button group for adjusting a setting applied to respective items of plot data or performing an operation on respective items of plot data.

For example, as illustrated in the same figure, this toolbox may include one, two, or three of
an area 233 that includes one or more buttons for performing an operation on plot data (for example, includes a create new plot button, a change plot format button, a copy plot data button, and a paste plot data button (for overlay analysis), etc.);
an area 234 that includes one or more buttons for adjusting a plot data axis (for example, a button for changing a scale setting (log scale or linear scale) for each axis, an automatic adjustment button for both axes, an axis copy/paste button, an axis adjustment tool button, etc.); and
an area 235 that includes one or more buttons for exporting a worksheet.

The first user uses this toolbox to perform a setting for each plot. As a result, plot setting data is generated. In other words, the plot setting data includes any one or more items of data inputted via the toolbox. Data inputted in such a manner may be included as a portion of the operation control data, or included as a portion of the “(b) display control data pertaining to display for the processing execution operation screen in the second mode,” for example. For example, axis specification data that specifies a display format for plot data axes (data inputted via the area 234) may be included in the display control data in (b) above. Note that these items of data may be handled as one that is included in the “(a) processing condition data employed by a processing apparatus that executes the processing in the second mode.”

### (Gate Toolbox)

FIG. 19 illustrates an example of a toolbox displayed by the gate toolbox display button being selected. A toolbox 236 indicated in the same figure includes a button group for setting a gate to respective items of plot data.

For example, as illustrated in the same figure, this toolbox may include one, two, or three of
an area 237 that includes one or more buttons for performing an operation on each gate (for example, includes a create new gate button, a change gate format button, a copy gate button, a paste gate button, a delete gate button, etc.);
an area 238 that includes one or more buttons for changing a display format for each gate (for example, includes a button for changing the color of a line that defines a gate, a button for changing the width of a line, a button for changing a forward/backward relation for a group of gates that mutually overlap, a change setting button for statistical processing with respect to an event included in each gate, etc.); and
and area 239 that includes a button for exporting data (statistical data, etc.) pertaining to each gate.

The first user uses this toolbox to perform a setting for each gate. As a result, gate setting data is generated. In other words, the gate setting data includes any one or more items of data inputted via the toolbox. Data inputted in such a manner may be included as a portion of the operation control data, or included as a portion of the “(b) display control data pertaining to display for the processing execution operation screen in the second mode,” for example. For example, the gate specification data that specifies a generated gate (data inputted via the area 237), the data that specifies the display format of a gate (data inputted via the area 238), or both of these may be included in the display control data in (b) above. Note that these items of data may be handled as one that is included in the “(a) processing condition data employed by a processing apparatus that executes the processing in the second mode.”

### (Operation Panel Displayed Near Plot)

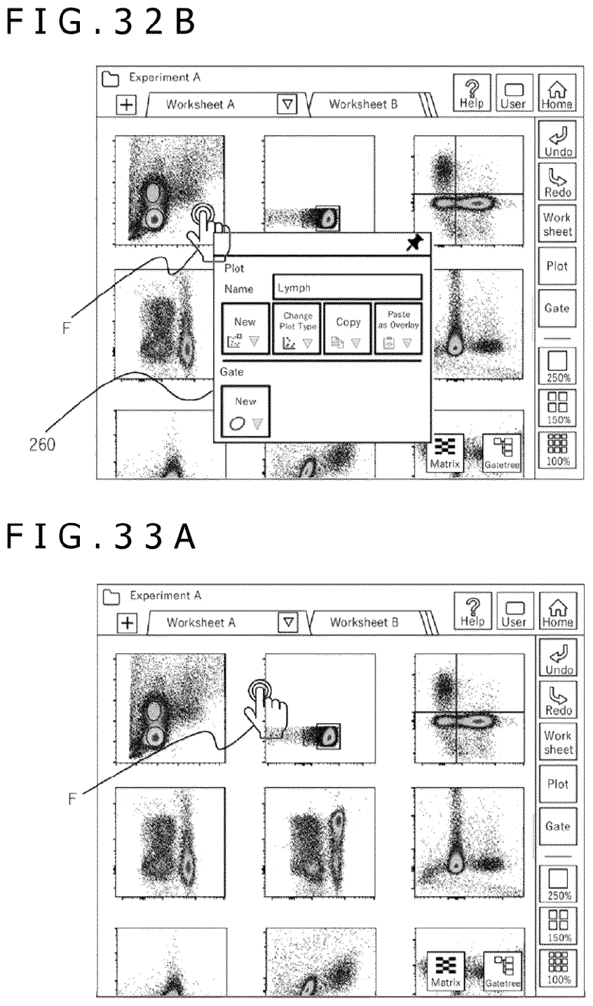

In a desirable embodiment of the present disclosure, in response to any plot in the second display area being touched, the information processing unit 102 causes an operation panel for performing an operation on the plot to be displayed near the touched position. An example of this operation panel is illustrated in FIGS. 32A and 32B.

As illustrated in FIG. 32A, multiple plots are displayed in the second display area. It is assumed that a finger F of the first user has touched any plot from among these. In response to this plot being touched, the information processing unit 102 causes an operation panel 260 to be displayed near the plot, as illustrated in FIG. 32B. It may be that the position at which the operation panel is displayed is, more specifically, near the touched position.

The information processing unit 102 may cause the operation panel 260 to be displayed, for example, within 5 cm from the touched position, desirably, within 4 cm, more desirably, within 3 cm, and yet more desirably, within 2 cm. This distance may mean the distance between the touched position and a point that is closest to the touched position within the area of the operation panel.

The operation panel may include one or more operation tool selection buttons. These operation tool selection buttons may include one or more tool buttons having a high frequency of use by a user, for example. Desirably, an operation tool selection button included in the operation panel may be configured to be able to be changed by a user. These operation tool selection buttons, for example, include any one or more buttons included in the plot toolbox and/or any one or more buttons included in the gate toolbox. The operation panel 260 indicated in FIG. 32B includes a create new plot button, a change plot format button, a copy plot data button, and a paste plot data button among the buttons described in relation to the plot toolbox, and includes a create new gate button among the buttons described in relation to the gate toolbox.

In addition, it may be that, according to a touched plot portion, a different operation panel is displayed near the touched position. For example, in response to an X axis portion of a plot being touched, the information processing unit 102 may cause an operation panel (toolbox) for adjusting the X axis to be displayed near the touched position. In addition, in response to a Y axis portion of a plot being touched, the information processing unit 102 may cause an operation panel (toolbox) for adjusting the Y axis to be displayed near the touched position. Furthermore, in response to a portion within a gate frame being touched, the information processing unit 102 may cause an operation panel (toolbox) for adjusting the gate to be displayed near the touched position.

### (Axis Operation Slider Bar)

In a desirable embodiment, in response to an axis of any plot in the second display area being touched, the information processing unit 102 causes a slider bar for changing the display format of this axis to be displayed. The slider bar is for adjusting a numerical range or a scale for each axis. The axes of each plot data (X axis and Y axis) may be axes in format from one of Biexponential, Linear, or Log.

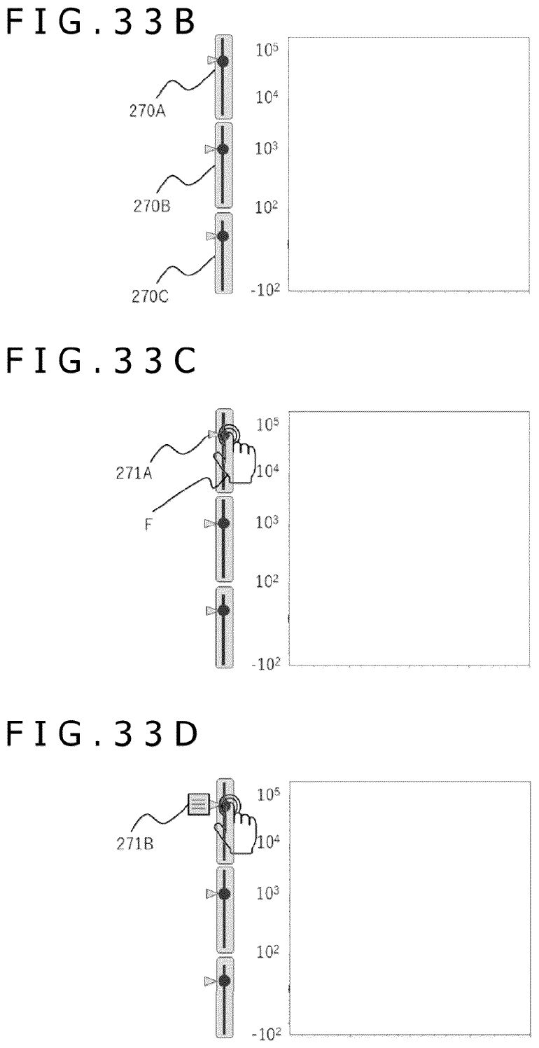

For example, three slider bars are displayed for a Biexponential axis setting. An example of slider bars displayed for a Biexponential axis setting is described with reference to FIGS. 33A through 33D.

As illustrated in FIG. 33A, the first user touches an axis portion of one item of plot data. In response to the axis portion being touched by the finger F of the user, as illustrated in FIG. 33B, the information processing unit 102 causes three slider bars 270A, 270B, and 270C to be displayed to the side of this axis for the plot data. Among these, the slider bar 270A is used to adjust the maximum value for the axis, the slider 270B is used to adjust a width near 0 for the axis, and the slider bar 270C is used to adjust the minimum value for the axis. In response to a drag operation on a knob in a respective slider bar, the information processing unit 102 changes the plot data (axis and display of event data). Note that change of the plot data may be continued even in a case where the drag operation is for movement upward or downward beyond a display range for the slider bar. As a result, it is easy to adjust the axis even in a case where plot data is displayed small, for example. The abovementioned three types of slider bars are displayed in FIG. 33B, but the information processing unit 102 may cause any one or two of these three types to be displayed.

Note that the information processing unit 102 removes the sliders in response to a region other than a slider bar being touched.

As illustrated in FIG. 33C, the first user touches any slider bar. In response to this slider bar being touched by the finger F of the user, as illustrated in FIG. 33D, the information processing unit 102 changes a display format for a knob 271 belonging to the slider bar. As illustrated in FIGS. 33C and 33D, this change is desirably such that a knob 271B available after the touch becomes larger than a knob 271A available before the touch, and/or the shape of the knob 271B available after the touch differs from the shape of the knob 271A available before the touch.

In addition, one slider bar may be displayed for a Linear axis or a Log axis setting. The information processing unit 102 may change a numerical range for an axis in response to a knob on this one slider bar being moved.

By employing slider bars such as the above, it is possible to improve operability of an axis operation by a touch.

### (Display of Enlarged Image)

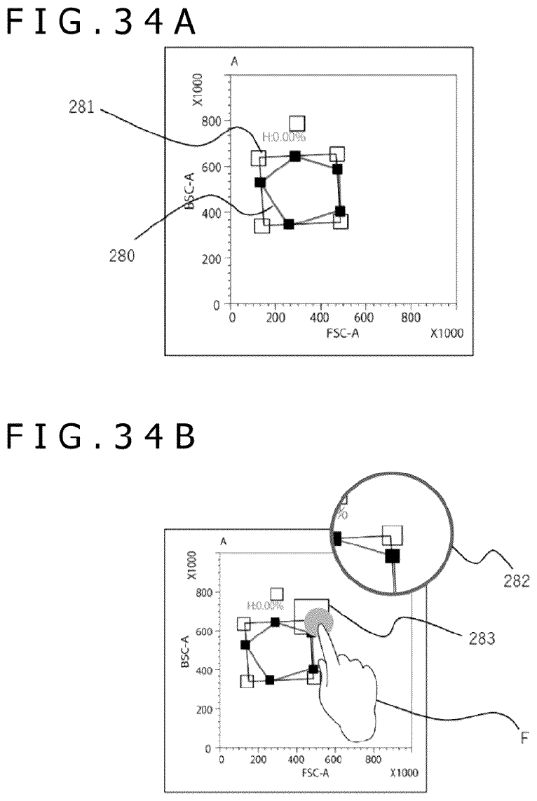

In response to any plot data display portion in the second display area being touched, the information processing unit 102 can cause an enlarged image of the touched portion to be displayed. Desirably, in response to any gate (specifically, a handle set to the gate) set for plot data being touched by a user, the information processing unit 102 causes an enlarged image of the touched position to be displayed near this position. With reference to FIGS. 34A and 34B, description is given below regarding display of this enlarged image.

A gate 280 is set for the plot data illustrated in FIG. 34A. Moreover, multiple handles 281 (indicated by quadrilaterals) are also set in advance to the gate 280. When one handle is touched by the finger F of the user, in response to the handle being touched, as illustrated in FIG. 34B, the information processing unit 102 displays an enlarged image 282 centered on the position of the handle to be displayed near the touched position or near the plot data. In addition, in conjunction with display of the enlarged image 282, the information processing unit 102 displays the touched handle larger, as indicated by a reference symbol 283.

In addition, the entire gate is selected in response to a portion of the gate 280 other than a handle (line that defines the gate or the inside thereof) being touched, and the information processing unit 102 causes the entire gate to move according to a drag operation, for example.

The shape of each handle may be a polygon (specifically, a rectangle) or a round shape, or may be another shape. It may be that the size of the size of each handle (long side for a rectangle, one side for a square, diameter for a circle) is, in a state of not being touched, for example, 5 to 25 dots and desirably 10 to 20 dots and is, in a state of being touched (state of being displayed large as described above), for example, 30 to 60 dots and desirably 40 to 55 dots. In addition, it may be that the size of a handle in a state of being touched is 1.5 to 5 times the size of the handle in a state of not being touched, and desirably 2 to 4 times. By displaying a touched handle large in such a manner, it is easier for a user to identify a touched handle.

There is a wide variety of content to be displayed within the processing condition setting screen. As a result, there are many cases in which the size of a region in which plot data is displayed is limited and, in conjunction with this, each item of plot data is displayed small. However, precisely adjusting a gate set to plot data is required. By an enlarged image being displayed as described above, precise gate adjustment becomes possible, even in a case in which plot data is displayed small.

### (Sorting Step S104)

For example, in response to the first user selecting the Sort button after setting of gate information for sorting is completed in the abovementioned Analysis step, the bioparticle sorting apparatus 100 changes the color of the button in the first display area (specifically, the procedure button area) to indicate a state where the Sort button is selected.

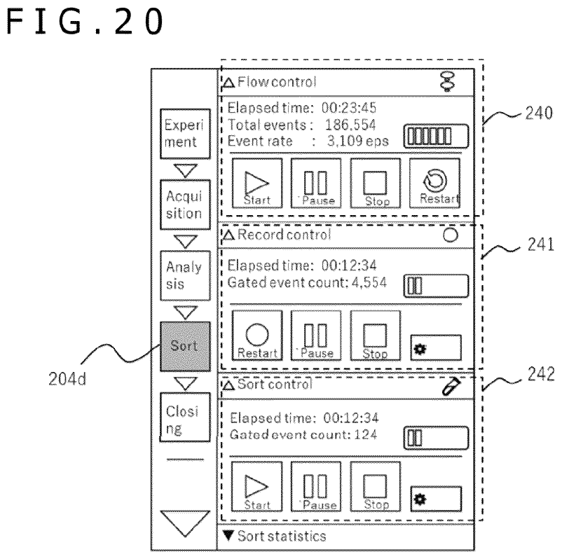

In response to the first user selecting the Sort button, the bioparticle sorting apparatus 100 displays, in the setting information display area, a screen for the bioparticle sorting apparatus 100 to control a sorting process, as illustrated in FIG. 20. In a state where the Sort button is selected, the bioparticle sorting apparatus 100 causes a flow control area 240, a recording control area 241, and a sorting control area 242 to be displayed in the setting information display area.

The flow control area and the recording control area are the same as that described in relation to the Acquisition step above.

As illustrated in the same figure, the sorting control area includes a start button (Start button) for starting a sorting process, a pause button (Pause button) for pausing the sorting process, and an end button (Stop button) for ending the sorting process. In addition, in the sorting control area, an elapsed amount of time for the sorting operation (Elapsed time), the number of times sorting has been executed in the sorting operation (Gated event count), etc., may be displayed.

### (Template Creation Step S105)

In response to a user selecting a Template button 204g at any timing, the bioparticle sorting apparatus 100 changes the color of the button in the first display area (specifically, the procedure button area) to indicate a state in which the Template button is selected. In FIG. 11, a template creation step S105 is indicated as being executed after processing in the sorting step S104, for example, but the template creation step S105 may be executed after any other step (S101, S102, S103, or S105).

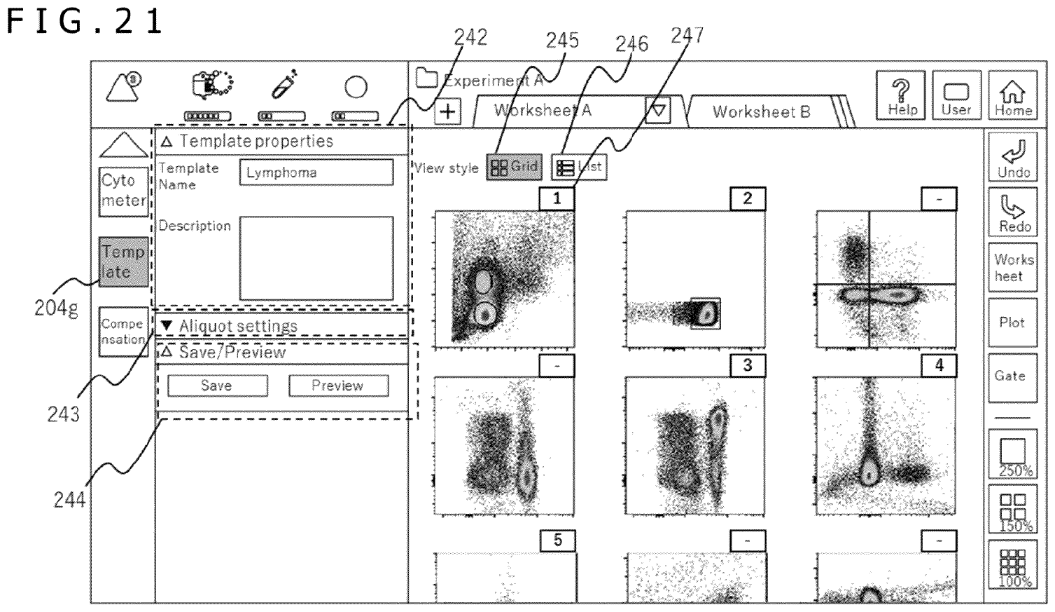

In response to a user selecting the Template button, the bioparticle sorting apparatus 100 displays a template property area 242, as illustrated in FIG. 21, in the setting information display area. The template property area includes a template name input field (Template name field) and a field for inputting a description regarding a template (Description field). In addition, the template property area may, for example, display the number of revisions and/or a creation date, etc., for the template.

The first user inputs template property data (a template name and/or a description regarding the template) in the template property area. As a result, the template property data is generated. In other words, the template property data includes any one or more items of data inputted via the template property area. Data inputted in such a manner may be included in the operation control data, or specifically may be included as a portion of the “(c) operator-directed instruction data that is directed at an operator who operates the processing apparatus in the second mode.” For example, template specification data that includes a template name and/or a description regarding a template may be included in the operator-directed instruction data in (c) above. In addition, the template specification data may also include the number of revisions and/or a creation date, etc., for the template.

In addition, as illustrated in the same figure, the setting information display area also displays an aliquot setting button 243. By this button being selected, a later-described aliquot setting area is deployed within the setting information display area. This aliquot setting area may be deployed in advance in the setting information display area.

In the present specification, an aliquot (Aliquot) means a sample (specifically, a bioparticle-containing sample) sorted up until a pause is performed, a sorting process being paused at a predetermined timing partway through the sorting process. For example, an aliquot is added to analysis by an apparatus or analysis kit that is separate from the bioparticle sorting apparatus 100 or to an apparatus or analysis kit that is attached to the bioparticle sorting apparatus 100. The sorting process is resumed in a case where the aliquot satisfies a predetermined condition, as a result of the analysis. By obtaining and analyzing the aliquot, it is possible to check whether bioparticles obtained by the sorting process satisfy a predetermined condition, and it is possible to determine whether to continue the sorting process or to adjust the sorting condition. In addition, it is also possible to avoid executing a wasteful sorting process.

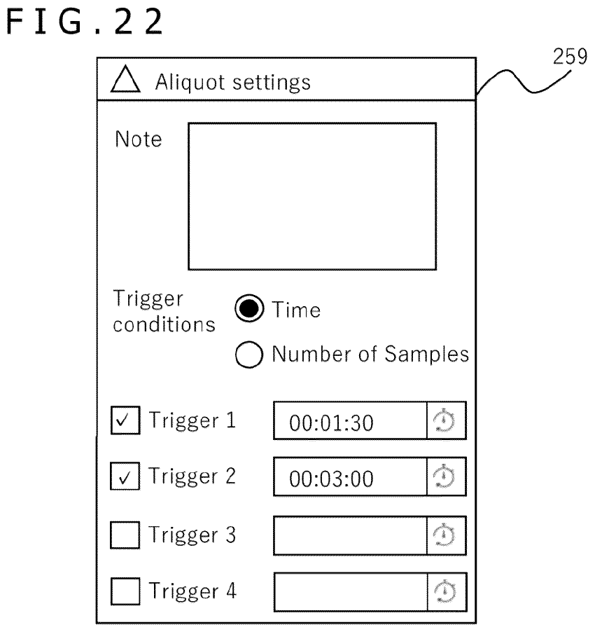

FIG. 22 illustrates an example of a screen for an aliquot setting area. An aliquot setting area 259 illustrated in the same figure includes a field for inputting an operator-directed instruction pertaining to aliquot obtainment and a timing setting field for setting one or more timings at which to obtain an aliquot. The timing setting field includes a field for selecting whether to obtain an aliquot at a specific timing or whether to obtain an aliquot with a specific number of samples and a field for specifying the timing or number of samples.

The first user inputs aliquot setting data (for example, aliquot obtainment instruction data that includes an operator-directed instruction pertaining to aliquot obtainment and/or timing setting data for setting the timing) to the aliquot setting area. As a result, aliquot setting data is generated. In other words, aliquot setting data includes any one or more items of data that have been inputted via the aliquot setting area. Data inputted in such a manner may be included as a portion of the operation control data or, for example, may be included as a portion of the “(a) processing condition data employed by a processing apparatus that executes the processing in the second mode” or the “(c) operator-directed instruction data that is directed at an operator who operates the processing apparatus in the second mode.” For example, the aliquot obtainment instruction data may be included in the operator-directed instruction data in the above (c). In addition, the timing setting data may be included in the processing condition data in (a) above.

In a state where the Template button is being selected, the second display area displays a grid display button 245 for disposing multiple items of measurement data (specifically, plot data) in a grid shape and a list display button 246 for disposing the multiple items of measurement data in a list shape. Note that, at a timing when the Template button is selected, one of the grid display button or the list display button may be selected by default.

In response to a user selecting the grid display button, the bioparticle sorting apparatus 100 causes an order designation field 247 for designating an adjustment order in the second display mode to be displayed near each item of measurement data, as illustrated in FIG. 21. The order designation fields may be displayed to be in contact with respective items of measurement data, as illustrated in the same figure. By order data being inputted to the order designation fields, it is possible to control an order for plot data operated by the second user in the second display mode, and the second user can perform gate adjustments in an appropriate order.

For example, the order designation fields may be configured such that any positive integer can be selected, or configured such that any positive integer can be inputted. The positive integers correspond to the adjustment order. For example, in the same figure, among the three items of plot data in the first row, “1” is selected for the plot data on the left, and “2” is selected for the plot data in the middle.

In addition, there are cases where there is measurement data that does not need to be displayed in the second display mode. To handle this case, the order designation field may be configured such that it is possible to select an option indicating display is unnecessary, such as “-” or “display unnecessary,” for example. In FIG. 21, for example, “-” indicating display is unnecessary is selected for plot data on the right among the three items of plot data in the first row. Note that the configuration may be made such that an option indicating display is necessary can be selected in the order designation field.

In the order designation field for each item of measurement data (plot data) displayed in the second display area, the first user inputs or selects an adjustment order or inputs or selects the necessity of display (specifically, display unnecessary). As a result, measurement data display setting data is generated. In other words, the measurement data display setting data includes data inputted via the order designation fields in such a manner, and, for example, includes adjustment order data or display necessity data. The generated measurement data display setting data may be included as a portion of the operation control data, or specifically may be included as a portion of the “(b) display control data pertaining to display for the processing execution operation screen in the second mode.” Note that measurement data display setting data may be handled as one that is included in the “(c) operator-directed instruction data that is directed at an operator who operates the processing apparatus in the second mode.” Such measurement data display setting data is useful to accurately convey an instruction pertaining to a sorting process to a user in the second display mode.

In addition, measurement data for which an adjustment order has been designated in the order designation field, measurement data for which display being necessary has been designated, or both of these may be included as a portion of the operation control data. Alternatively, image data for these items of measurement data may be included as a portion of the operation control data.

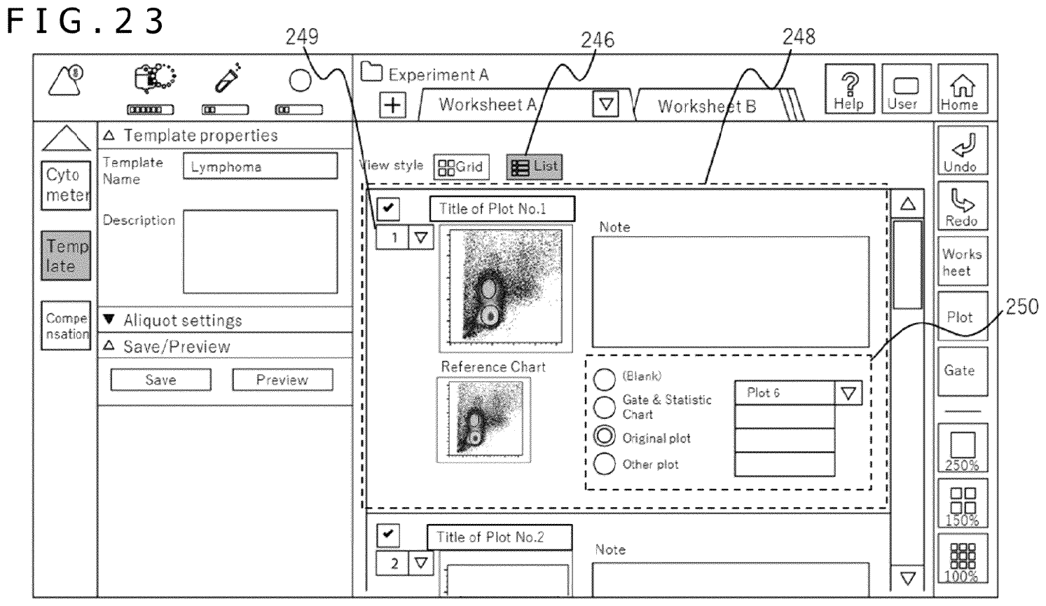

In response to a user selecting the list display button 246, the information processing apparatus 102 causes one or more operator-directed instruction data input areas 248 that each include one item of measurement data and various instruction data input fields pertaining to the measurement data to be displayed, as illustrated in FIG. 23.

Each operator-directed instruction data input area may display an order designation field 249 for designating an adjustment order in the second display mode for measurement data included in each area. The order designation field is similar to that described above.

The operator-directed instruction data input area includes a memo input field (Note field) for inputting a memo (specifically, an instruction memo) directed at an operator and/or a selection area 250 for selecting data that an operator should refer to.

The memo inputted to the memo input field is displayed accompanying the measurement data when the measurement data is displayed in the second display mode. As a result, the second user can check an instruction from the first user.

The first user inputs, in the memo input field, a memo directed at the second user. As a result, memo data is generated. The generated memo data may be included in the operation control data, or specifically may be included as a portion of the “(c) operator-directed instruction data that is directed at an operator who operates the processing apparatus in the second mode.” The memo data is useful to accurately convey an instruction from a user in the first display mode to a user in the second display mode.

In a case where measurement data that should be referred to (hereinafter may also be referred to as reference measurement data) is selected in the selection area, when measurement data is displayed in the second display mode, the reference measurement data is displayed accompanying the measurement data. As a result, the second user can adjust a sorting condition while referring to the reference measurement data.

The selection area may include, as options, one, two, three, or all four of “no data that should be referred to,” “list of gates,” “ideal measurement data,” and “other measurement data (plot data),” for example.

The first user selects reference measurement data in the selection area. As a result, the reference measurement data is generated. The reference measurement data may be included as a portion of the operation control data, or specifically may be included as a portion of the “(c) operator-directed instruction data that is directed at an operator who operates the processing apparatus in the second mode.” The reference measurement data is useful for adjusting a processing condition for a sorting process in the second display mode.

Each operator-directed instruction data input area may display data that should be referred to (Reference Chart field) and has been selected in the selection area. In addition, the selection area may display a list box (in the same figure, a box for which Plot 6 is displayed is the list box) for selecting data that should be referred to and has been selected in the selection field (specifically, ideal measurement data or other measurement data).

In addition, data that can be inputted to the operator-directed instruction data input area is not limited to that described above. For example, the operator-directed instruction data input area may include an area for specifying a gate operation that can be executed by the second user. For example, it is possible to give, as examples of gate operations, moving the position of a gate, changing the size of a gate (enlarging or shrinking a gate), and changing the shape of a gate. As more specific examples of the abovementioned gate shape change, it is possible to give changing the inclination of a gate, changing the diameter or axis of a gate (for example, changing the length of the diameter of a circular gate or changing the length of the long axis or/and short axis of an oval-shaped gate), changing a ratio for a diameter or an axis of a gate (for example, changing a circular gate to an oval-shaped gate or the reverse, or changing the ratio for the lengths of the long axis and short axis of an oval-shaped gate), and changing the eccentricity or oblateness of a gate. For example, the first user specifies a gate operation in this area, whereby the information processing unit 102 generates gate operation limit data. In reference to the gate operation limit data, the information processing apparatus 102 limits some gate operations by the second user in the second display mode. As a result, it is possible to prevent an unnecessary gate adjustment from being executed in the second display mode.

Note that, in a case where an area for specifying a gate operation is not displayed, the information processing unit 102 may hold gate operation limit data in advance, and, for example, the gate operation limit data may be data for limiting any gate operation (for example, changing the size of a gate and/or changing the shape of a gate, etc.) according to the second display mode. In one embodiment of the present disclosure, gate operation limit data may be operation limit data for limiting changes to the shape of a gate or, for example, may be data that limits changes to the shape of a gate but does not limit changes to the size of a gate. In addition, it may be that gate operation limit data can be inputted in another area (such as the first display area), for example.

The bioparticle sorting apparatus according to the present disclosure as above causes a screen for inputting measurement data (specifically, plot data) obtained using a predetermined gate setting and operator-directed instruction data regarding measurement data obtained using this gate setting to be displayed. In the present specification, this screen is referred to as a template generation screen. The bioparticle sorting apparatus according to the present disclosure is configured such that operation control data can be inputted via the template generation screen, and, as a result, convenience is improved for the first user to generate instruction data directed at the second user. Moreover, the bioparticle sorting apparatus according to the present disclosure, in reference to the operation control data, displays a processing execution operation screen directed at the second user in the second display mode which is described later. A processing execution operation screen based on the operation control data is displayed, whereby it is easier to perform an adjustment or an operation when the second user performs sorting.

In addition, as illustrated in the same figure, the setting information display area displays an area 244 that includes buttons for saving or previewing generated operation control data. This area displays a save button (Save button) for saving operation control data and a preview button (Preview button) for previewing a screen (specifically, a screen that is displayed in the second display mode) that is generated in reference to the operation control data. The information processing unit 102 executes the respective abovementioned function in response to the respective button being selected.

### (Closing Step S105)

For example, in response to the user selecting the Closing button after the sorting process is completed in the abovementioned Sort step, the bioparticle sorting apparatus 100 changes the color of the button in the first display area (specifically, the procedure button area) to indicate a state in which the Closing button is selected.

In response to a user selecting the Closing button, the bioparticle sorting apparatus 100 displays, in the setting information display area, a screen for ending setting of a sorting process condition or a screen for ending the first display mode. These screens may display, inter alia, a button for closing an open experiment file and a log out button for ending the first display mode.

### (Cytometer Step)

Processing in the first display mode may optionally include a cytometer step. The cytometer step is for adjusting a setting for an apparatus.

For example, in response to the user selecting the Cytometer button at any timing in processing in the first display mode, the bioparticle sorting apparatus 100 changes the color of the button in the first display area (specifically, the procedure button area) to indicate a state where the Cytometer button is selected. In response to the user selecting the Cytometer button, the bioparticle sorting apparatus 100 can display, in the setting information display area, a screen for adjusting settings for the apparatus. The bioparticle sorting apparatus 100 may be configured to accept adjustments to apparatus settings via this screen.

### (Correction Step)

Processing in the first display mode may optionally include a correction step. The correction step is for adjusting a fluorescence correction condition.

For example, in response to the user selecting a Compensation button at any timing in processing in the first display mode, the bioparticle sorting apparatus 100 changes the color of the button in the first display area (specifically, the procedure button area) to indicate a state where the Compensation button is selected. In response to the user selecting the Compensation button, the bioparticle sorting apparatus 100 can display, in the setting information display area, a screen for adjusting settings for fluorescence correction. The bioparticle sorting apparatus 100 may be configured to accept adjustments to fluorescence correction settings via this screen.

In addition, although it is described above that each step is executed in the order in which the steps are lined up in the procedure button area, an order for executing each step may be freely selected by a user. For example, any previous step may be returned to after the completion of a certain step, such as returning to the Acquisition step again after the Sort step. In addition, another step may be advanced to after skipping one or more steps, such as advancing to the Template step after the Analysis step. In addition, one or more steps may be omitted, such as the Compensation step being omitted.

The first user executes processing in each step as above, whereby operation control data is inputted. The information processing unit 102 saves the inputted operation control data. After the saving, the bioparticle sorting apparatus 100 may end the operation in the first display mode.

### (Operation Control Data)

Items of operation control data inputted in the group of steps described above are summarized below, for example.

As described above, the operation control data may include one or more of

As described above, the operation control data may include one or more of
(a) processing condition data employed by a processing apparatus that executes the processing in the second mode (hereinafter, may be referred to as “(a) data”),
(b) display control data pertaining to display for the processing execution operation screen in the second mode (hereinafter, may be referred to as “(b) data”), and
(c) operator-directed instruction data that is directed at an operator who operates the processing apparatus in the second mode (hereinafter, may be referred to as “(c) data”).

For example, the operation control data includes at least the (a) data, and more desirably, includes the (a) data and the (b) data or includes the (a) data and the (c) data.

Alternatively, the operation control data includes at least the (b) data, and more desirably, includes the (b) data and the (a) data or includes the (b) data and the (c) data.

Alternatively, the operation control data includes at least the (c) data, and more desirably, includes the (c) data and the (a) data or includes the (c) data and the (b) data.

In a desirable embodiment, the operation control data may include the (a) data, the (b) data, and the (c) data.

Using the operation control data that includes such data, it is possible for a user who uses the apparatus in the second display mode to generate a more appropriate processing execution operation screen.

The (a) data is processing condition data that the bioparticle sorting apparatus 100 employs in a sorting process in the second display mode. The (a) data may include instruction data directed at the bioparticle sorting apparatus, such as a gate setting, for example.

The (a) data includes data for identifying bioparticles that should be sorted in the second display mode, and more specifically, includes gate information for identifying the bioparticles that are to be sorted in the sorting process. For example, this data may specify a range for light data arising from the bioparticles that should be sorted. For example, the processing condition data may include data for stipulating a range, in one or more items of plot data, for bioparticles that are a sorting target. The processing condition data may be set, as appropriate, by a person skilled in the art, or may be set, as appropriate, by a user (such as a researcher or a developer, for example) who uses the bioparticle sorting apparatus 100 in the first display mode.

The (a) data may include data pertaining to an apparatus setting that the bioparticle sorting apparatus 100 employs in a sorting process. For example, data pertaining to settings for the light application unit, the detection unit, and the sorting unit may be included. Data pertaining to a setting for the light application unit may specify a type of light that should be applied. Data pertaining to a setting for the detection unit may include data that pertains to an operation setting for a detector and/or pertains to specifying a detector that operates. Data pertaining to a setting for the sorting unit may include data pertaining to a pressure and/or electric charge employed for the purpose of a sorting operation, or data pertaining to a voltage applied for the purpose of a sorting operation.

The (a) data may include one or more items of data described as data that may be included (or may be handled) as the (a) data in the description above. For example, the (a) data may include any one or more items of the setting data pertaining to the gain and/or threshold, the setting data pertaining to the correction matrix, the adjusted correction matrix data, the gate information, and the timing setting data.

The (b) data is display control data pertaining to display for the processing execution operation screen, which is displayed in the second display mode. For example, the (b) data may include data pertaining to control of the display of plot data displayed in the second display mode, and more specifically, may include one or more items of data pertaining to the display position of the plot data, data pertaining to an adjustment order for the plot data, and data pertaining to specifying displayed plot data.

The (b) data may include one or more items of data described as data that may be included (or may be handled) as the (b) data in the above description. For example, the (b) data may include any one or more items of the worksheet setting data, the plot setting data, the gate setting data, the gate operation limit data, and the measurement data display setting data.

It is particularly desirable that the (b) data include data pertaining to an operation limit for plot data displayed in the second display mode. This data pertaining to an operation limit may include data pertaining to a prohibition of changing the plot data per se. For example, it may be that, by the data pertaining to a prohibition, a change of the axis of a plot and/or a change of a display range for a plot is prohibited. As a result, it is possible to prevent a sorting process intended by a user in the first display mode from ceasing to be executable due to a user in the second display mode changing plot data.

The (c) data is operator-directed instruction data that is directed at an operator who operates the bioparticle sorting apparatus in the second display mode. Using the operator-directed instruction data, it is possible to convey an instruction by a user in the first display mode to a user in the second display mode.

For example, the (c) data may be inputted for each processing step in the second display mode. As a result, it is possible to convey, to a user in the second display mode, work that should be executed in each processing step.

The (c) data may be inputted for each item of plot data, or more specifically, may be a gate adjustment instruction that is inputted for each item of plot data. By the gate adjustment instruction, it is possible to have an appropriate gate setting for each sample.

The (c) data may include reference data pertaining to checking and/or adjusting the processing condition data. This reference data may include, for example, (c1) specification data for specifying plot data changed by the adjusting, (c2) checking graph data for the operator to refer to, (c3) statistical data pertaining to each gate, and (c4) image data associated with the processing condition data.

The (c1) specification data is, for example, data that associates plot data that is an adjustment target with plot data that is changed in conjunction with the adjustment. In response to the plot data that is the adjustment target being adjusted, the bioparticle sorting apparatus 100 (specifically, the information processing unit) can, in reference to the specification data, specify plot data to be changed in conjunction with the adjustment and adjust the plot data. As a result, the bioparticle sorting apparatus 100 (specifically, the information processing unit) can execute a plot data adjustment in real time according to the adjustment, in the second display mode, for example.

For example, the (c2) checking graph data is reference plot data that should be referred to when an operator adjusts a gate that has been set for a certain item of plot data, in the second display mode. The bioparticle sorting apparatus 100 (specifically, the information processing unit) causes the reference plot data to be displayed, whereby an operator can check whether sorted particles or data is one that is desired, in the second display mode in particular.

The (c3) statistical data pertaining to each gate is, for example, data pertaining to the number of events and/or a ratio included in each gate. The bioparticle sorting apparatus 100 (specifically, the information processing unit) causes the statistical data to be displayed, whereby an operator can check whether sorted particles or data is one that is desired, in the second display mode in particular.

For example, the (c4) image data associated with the processing condition data may be image data generated or processed as ideal data or data measured by another apparatus. In addition, the image data may be for indicating to an operator a countermeasure to be taken when abnormal data is obtained and, for example, may be an image indicating which portion of the bioparticle sorting apparatus 100 should be checked and/or operated. In addition, the image data associated with the processing condition data may be for giving an instruction to an operator. Using such image data, the bioparticle sorting apparatus 100 can give an instruction to an operator in the second display mode in particular.

The (c) data may include one or more items of data described as data that may be included (or may be handled) as the (c) data in the above description. For example, the (c) data may include any one or more items of the plot data, the template property data, the aliquot obtainment instruction data, the memo data, and the reference measurement data.

### (2-3) Second Display Mode

In the second display mode, the bioparticle sorting apparatus 100 displays a processing execution operation screen. The processing execution operation screen is for executing processing for sorting predetermined bioparticles. At least part of the processing execution operation screen may be generated in reference to operation control data inputted in the first display mode.

Desirably, the bioparticle sorting apparatus 100 controls display of the processing execution operation screen in the second display mode in reference to the operation control data. As a result, an intention of the first user regarding a sorting process is reflected to a sorting operation performed by the second user.

Desirably, in the second display mode, the operator can adjust the processing condition data following the operator-directed instruction data. A sorting process for bioparticles often requires fine adjustment for each sample. The bioparticle sorting apparatus 100 enables adjustment and thus enables fine adjustment.

Desirably, in the second display mode, adjustment of at least part of the gate information is limited. A condition that should not be adjusted by the second user is often included in sorting process conditions set by the first user. Accordingly, it is possible to limit adjustment in such a manner, whereby it is possible to prevent the second user from performing an undesirable processing condition adjustment.

### (2-3-1) Example of Configuration of Screen Displayed in Second Display Mode

### (Processing Execution Operation Screen)

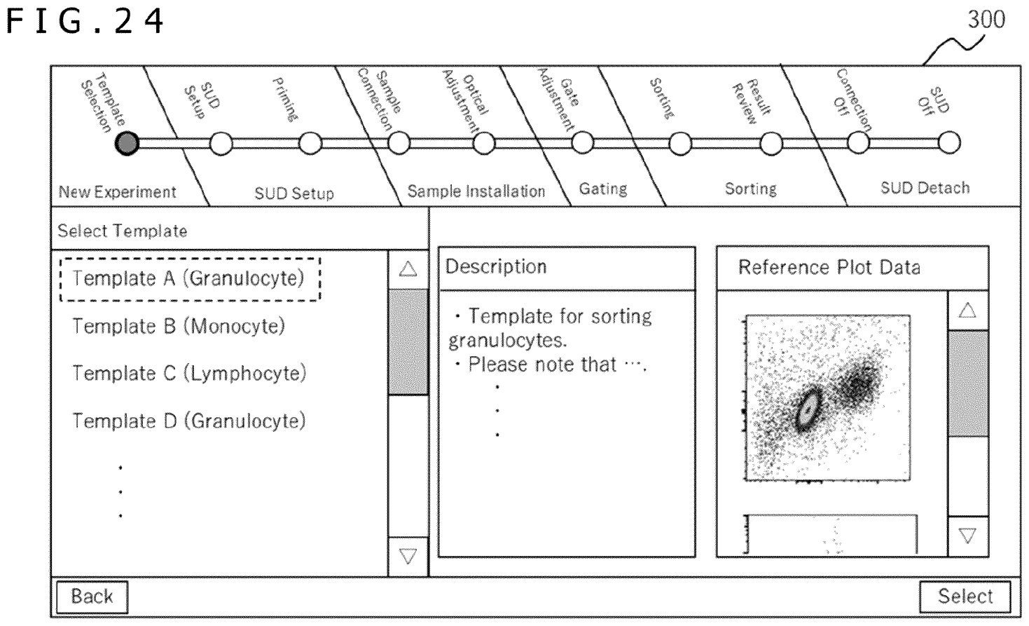

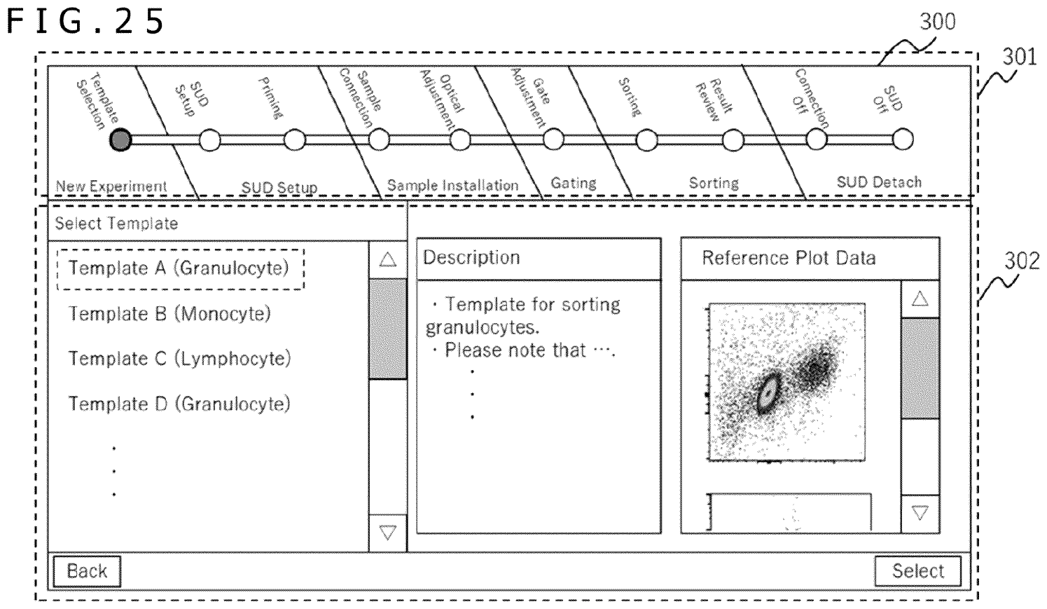

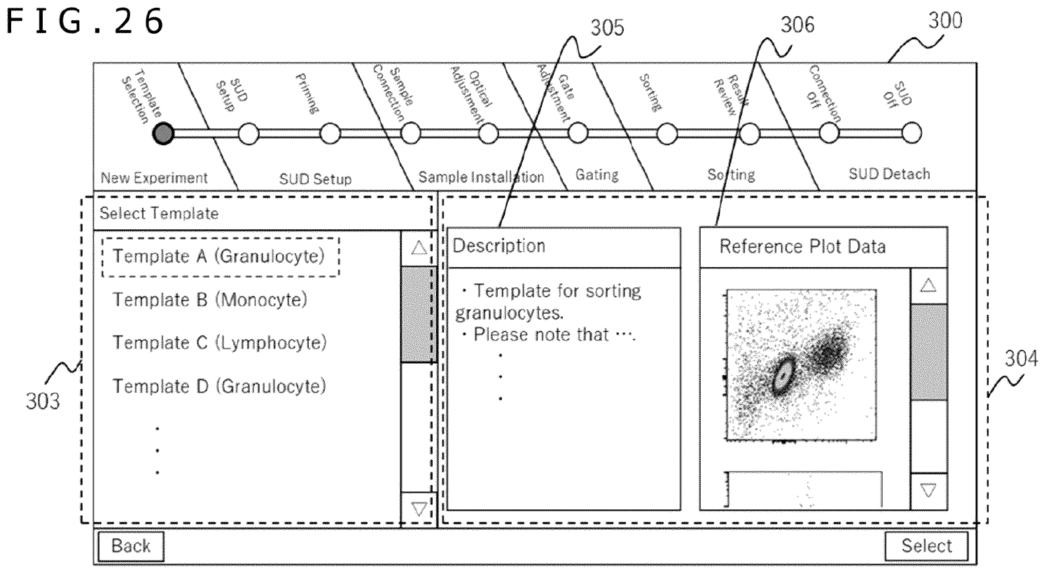

FIG. 24 illustrates an example of the processing execution operation screen that the bioparticle sorting apparatus 100 (specifically, the information processing unit 102) causes the display unit 101 to display in the second display mode. As illustrated in FIG. 25, a processing execution operation screen 300 illustrated in the same 24 includes a third display area (may also be referred to as a processing operation flow display area) 301 that displays an operation flow for a sorting process and a fourth display area (may also be referred to as an operation content display area) 302 that displays operation content for each step included in the processing operation flow.

As illustrated in the same figure, it may be that the third display area is disposed on the top side in the processing execution operation screen and the fourth display area is disposed on the bottom side in the processing execution operation screen.

Alternatively, it may be that the third display area is disposed on the bottom side in the processing execution operation screen and the fourth display area is disposed on the top side in the processing execution operation screen.

In the processing condition setting screen in the first display mode, two areas (the first display area and the second display area) are displayed divided into left and right, whereas, in the second display mode, two areas (the third display area and the fourth display area) are displayed divided into up and down as described above. In such a manner, by dividing and disposing the two main areas left and right in the first display mode and dividing and disposing the two main areas up and down in the second display mode, a user can clearly distinguish between operation modes for the bioparticle sorting apparatus.

In addition, it is envisioned that, compared to the first user, the second user has less knowledge pertaining to processing conditions for the bioparticle sorting apparatus. In addition, in order to prevent confusion arising in a sorting operation in the second display mode, it is desirable for only information necessary for the sorting operation to be displayed on the screen. In addition, it is desirable for this information to be displayed larger in order to prevent a sorting operation error. In the second display mode, disposing is performed by division between up and down as described above, whereby it is possible to display only necessary information larger, and this is useful to prevent a sorting operation error.

It may be that disposition of the third display area and the fourth display area can be changed. For example, the bioparticle sorting apparatus may be configured such that it is possible to change the screen from a state in which the former area is disposed on the top side and the latter area is disposed on the bottom side to a state in which the former area is disposed on the bottom side and the latter area is disposed on the top side. In addition, configuration may be taken such that it is possible to change the screen to achieve the opposite.

### (Third Display Area)

In the third display area 301, steps (and marks indicating the steps) to be executed in the second display mode are disposed aligned from left to right. In addition, indicating is also performed using a mark (circle marked filled with gray) that indicates processing that is being executed. In such a manner, in the present disclosure, it may be that steps to be executed in the second display mode are indicated and aligned in one direction in the third display area which is displayed in the second display mode. In addition, it may be that a mark indicating a step that is being executed is displayed at the position for the step, in the third display area.

### (Fourth Display Area)

Content displayed in the fourth display area 302 may be changed each step. The content includes, for example, description data pertaining to an operation that the second user should execute in respective steps and/or data indicating a status for the operation. At least some of this content may be generated in reference to operation control data inputted in the first display mode.

### (2-3-2) Example of Processing in Second Display Mode

### (Operation Start Process)

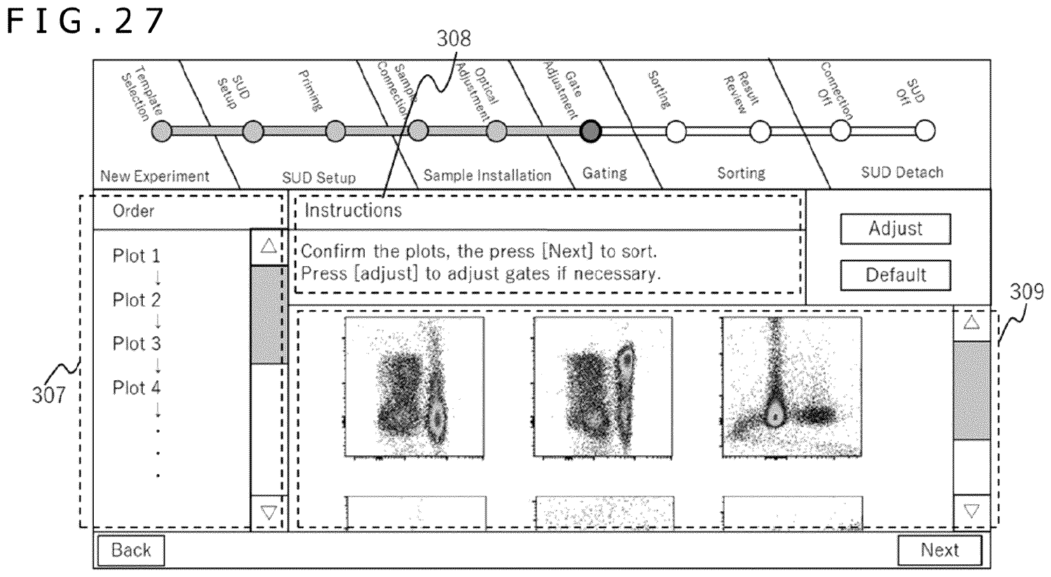

When processing is started in the second display mode, the bioparticle sorting apparatus 100 (specifically, the information processing unit 102) causes the display unit 101 to display the processing execution operation screen 300 illustrated in FIG. 26.

In the same figure, an operation start process is displayed as “New Experiment.” The operation start process includes a template selection step (displayed as “Template selection” in the same figure). The information processing unit 102 may cause an instruction mark (circle mark filled with gray) indicating processing that the bioparticle sorting apparatus 100 will execute to be displayed at a position for the template selection step, in the third display area. In such a manner, the bioparticle sorting apparatus 100 causes the instruction mark to be displayed at a position indicating a step that is being executed, among a series of steps, in the second display mode.

In addition, the information processing unit 102 causes a screen pertaining to operation content for the template selection step to be displayed in the fourth display area. For example, as illustrated in the same figure, the fourth display area displays a template selection field 303 and a template description field 304 that describes an outline of a template that is selected.

The information processing unit 102 displays a list of templates that can be selected in the template selection field. For example, it may be that this field displays a template name in reference to template property data, in particular, displays a template name that has been inputted to the template property area in the Template step in the first display mode. In the same figure, “Template A (Granulocyte)” is selected.

The information processing unit 102 causes a detailed description field 305 (displayed as Description in the same figure) pertaining to a selected template and a reference data field 306 (displayed as Reference Plot Data) that displays an example of measurement data (plot data) obtained in a case where a sorting process is executed according to the selected template to be displayed in the template description field.

The detailed description field displays a description pertaining to the selected template. For example, this field may display a description pertaining to a template that has been inputted to the template property area in the Template step in the first display mode. In other words, in reference to the template property data, the information processing unit 102 causes the detailed description field to display a description pertaining to a template. For example, as illustrated in the same figure, the detailed description field displays such a description as “template for sorting granulocytes” as a description pertaining to Template A.

The information processing unit 102 displays, in the reference data field, an example of measurement data (plot data) obtained in a case where a sorting process is executed according to a selected template. For example, an example of the measurement data may be measurement data, image data in particular, for which an adjustment order has been designated in the Template step in the first display mode. In such a manner, in reference to the operation control data, and in reference to image data included in the operation control data in particular, the information processing unit 102 causes measurement data to be displayed in the reference data field. For example, as illustrated in the same figure, the reference data field may display multiple items of plot data.

In response to a Select button disposed at the bottom-right of the processing execution operation screen being selected, the bioparticle sorting apparatus 100 advances the processing to the next step. In response to a Back button disposed at the bottom-right of the processing execution operation screen being selected, the processing returns to the previous step. These buttons may be displayed on another screen. Note that text in each button may be changed, as appropriate, to other text or a mark.

### (Setup Process)

In response to a template being selected in an operation start process, the bioparticle sorting apparatus advances the processing to a setup process. The setup process, for example, may include an attachment step (displayed as “SUD setup” in FIG. 24) for attaching a microchip for sorting bioparticles to the bioparticle sorting apparatus and a priming step (displayed as “Priming” in the same figure) for priming within a flow path of the microchip. Note that, in the same figure, SUD is the name of the microchip and another name may be written.

Steps included in the setup process and a screen displayed in each step may be changed, as appropriate, according to the type of the bioparticle sorting apparatus or the type of the attached microchip.

### (Sample Introduction Process)

After the setup process completes, the bioparticle sorting apparatus advances the processing to a sample introduction process. The sample introduction process, for example, may include a sample connection step (displayed as “Sample Connection” in FIG. 24) for connecting a container that includes a biological sample to be added to a sorting process to the bioparticle sorting apparatus and an optical adjustment step (displayed as “Optical Adjustment” in the same figure) for using a portion of the biological sample to perform a sorting operation and thereby perform an optical adjustment of the bioparticle sorting apparatus.

Steps included in the sample introduction process and a screen displayed in each step may be changed, as appropriate, according to the type of the bioparticle sorting apparatus.

The bioparticle sorting apparatus may execute these steps in reference to the operation control data (specifically, in reference to processing condition data).

### (Gating Process)

After the sample introduction process completes, the bioparticle sorting apparatus advances the processing to a gating process. As illustrated in FIG. 27, the gating process, for example, includes a gate adjustment step (displayed as “Gate Adjustment” in FIG. 27) for adjusting a gate set for respective plot data in the first display mode, with respect to measurement data.

In the gate adjustment step, the bioparticle sorting apparatus 100 may obtain plot data by performing light application and detection with respect to a portion of a sample. Obtainment of corresponding plot data is executed according to processing condition data inputted in the first display mode.

In the gate adjustment step, as illustrated in FIG. 27, the bioparticle sorting apparatus 100 causes an instruction mark indicating processing the bioparticle sorting apparatus 100 executes to be displayed at a position for the gate adjustment step, in the third display area. By causing the instruction mark to move to a position for each step in such a manner, the second user can understand the work status.

In addition, it may be that, in the third display area, completed processing is displayed differently from processing that is not complete. For example, in FIG. 27, completed processing is displayed in gray, and incomplete processing is displayed in white.

In addition, the information processing unit 102 causes a screen pertaining to operation content for the gate adjustment step to be displayed in the fourth display area. For example, as illustrated in the same figure, the fourth display area may include an adjustment order display field 307 (displayed as “Order” in the same figure), a field 308 for making an instruction to the second user, and a data display field 309.

In reference to the operation control data (specifically, measurement data display setting data, and more specifically, data pertaining to an adjustment order) inputted in the first display mode, the information processing unit 102 displays a list of items of plot data that are adjustment targets in the adjustment order display field, as illustrated in the same figure. In this list, the names of items of plot data may be displayed according to the adjustment order.

In reference to the operation control data (specifically, operator-directed instruction data) inputted in the first display mode, the information processing unit 102 displays an instruction to the second user in the field for making an instruction to the second user, as illustrated in the same figure.

As illustrated in the same figure, the information processing unit 102 displays, in the data display field, a plot data group generated by execution of a sorting process on a sample. In reference to the operation control data (specifically, display control data and more specifically more specifically, measurement data display setting data) that has been inputted in the first display mode, the information processing unit 102 may specify a plot data group that should be displayed. This plot data group, for example, may include one or more items of plot data corresponding to the list of items of plot data that are adjustment targets. This plot data group, for example, may further include one or more items of plot data that are not adjustment targets.

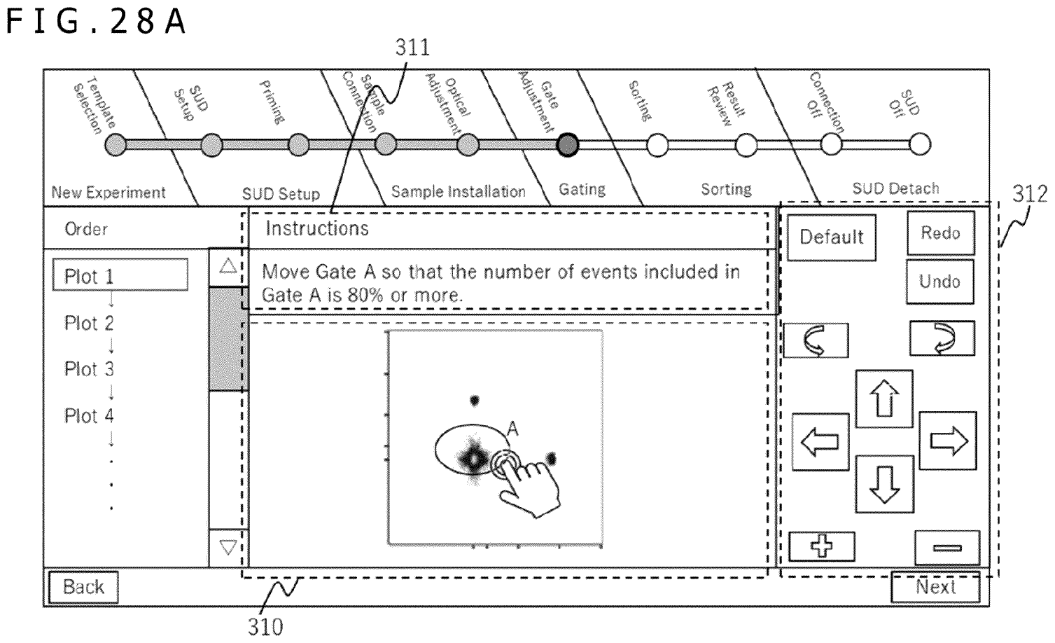

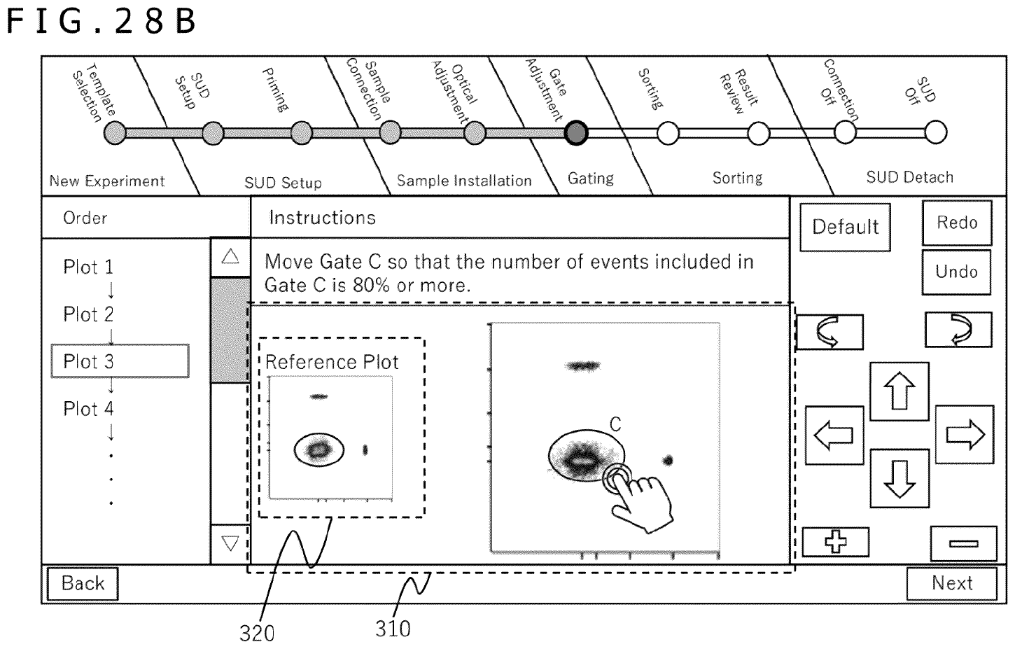

The second user checks the plot data group according to the instruction displayed in the instruction field. Next, the second user adjusts gates in the one or more items of plot data, according to the adjustment order. For this adjustment, the second user selects an item of plot data that should be adjusted first, from the plot data list displayed in the adjustment order display field. A state in which a selection has been made is indicated in FIG. 28A.

In response to the second user selecting the plot 1 which is plot data that should be adjusted first, the information processing unit 102 displays plot data for the plot 1 in a data display field 310, as illustrated in the same figure. A gate A set according to the processing condition data is also displayed in the plot data.

In response to the second user selecting plot 1 which is plot data that should be adjusted first, the information processing unit 102 displays an instruction pertaining to gate adjustment of the plot 1 in the instruction field 311, as illustrated in the same figure. The bioparticle sorting apparatus 100 may display the instruction in reference to the operation control data (specifically, operator-directed instruction data) inputted in the first display mode, and specifically, displays a memo (specifically, an instruction memo) that has been inputted in the memo input field. The second user can precisely execute the gate adjustment by referring to the instruction field.

In addition, it may be that, in response to the second user selecting the plot 1 which is plot data that should be adjusted first, the bioparticle sorting apparatus 100 causes a gate operation button field 312 to be displayed adjacent to the data display field.

This gate operation button field includes a “Default” button for returning the gate to a state before an adjustment by the second user is made, a “Redo” button for redoing an operation performed on a gate or performing a canceled operation again, and an “Undo” button for canceling an operation performed on a gate. In addition, the gate operation button field may include a group of buttons for performing operations on a gate position and/or a group of buttons for changing the size or shape of a gate.

The bioparticle sorting apparatus 100 may be configured such that some gate operations are limited in the second display mode. For example, configuration may be such that it is not possible to change one of or both the size or shape of a gate. The information processing unit 102 may limit a corresponding operation in reference to the operation control data. In one embodiment, in reference to the operation control data (specifically, display control data, and more specifically, gate operation limit data), the information processing unit 102 limits changing the shape of a gate but permits changing the size of a gate. For example, the information processing unit 102 can change the size of the gate A in response to the second user touching and dragging the gate A. For example, in response to the second user dragging, in a touched state, a specific position (for example, a line that defines the gate A or a size change mark that is displayed in the gate A or is displayed on a line that defines the gate A), the information processing unit 102 can change the size of the gate A.

In addition, the information processing unit 102 may, in reference to the operation control data, permit changing the position of a gate. For example, the information processing unit 102 can change the position of the gate A in response to the second user touching and dragging the gate A. For example, in response to the second user dragging, in a state of touching, in response to dragging, in a touched state) another specific position (for example, the center of the gate A, or any position change mark displayed in the gate A or displayed on a line that defines the gate A), the information processing unit 102 causes the position of the gate A to move in the direction of the dragging.

The second user completes a gate adjustment for the plot 1, subsequently selects the plot that should be adjusted next from the group of plots listed in the adjustment order display field, and then performs a gate adjustment that pertains to the selected plot. This gate adjustment may be executed in accordance with an operator-directed instruction included in operation control data, similarly for the plot 1.

FIG. 28B illustrates an example of a screen in which another item of plot data that should be adjusted is selected. In the same figure, a plot 3 is selected as plot data that should be adjusted.

In response to the second user selecting plot 3, the information processing unit 102 displays plot data for plot 3 in the data display field 310, as illustrated in the same figure. A gate C set according to the processing condition data is also displayed in the plot data.

In addition, it may be that, as illustrated in the same figure, the information processing unit 102 causes checking graph data (Reference Plot), which should be referred to in order to adjust the gate C, to be displayed in the data display field 310. The information processing unit 102 can cause the checking graph data to be displayed in reference to the operation control data (specifically, the operator-directed instruction data, and more specifically, reference data). As illustrated in the same figure, the checking graph data may include an image of plot data obtained in the first display mode and a gate that a user in the first display mode has set with respect to the plot data. Such checking graph data being outputted to a screen is useful for a user in the second display mode to precisely perform a gate adjustment in plot data.

In response to gate adjustment of one or more plots that are adjustment targets having all been completed, the bioparticle sorting apparatus 100 advances the processing to a sorting process.

### (Sorting Process)

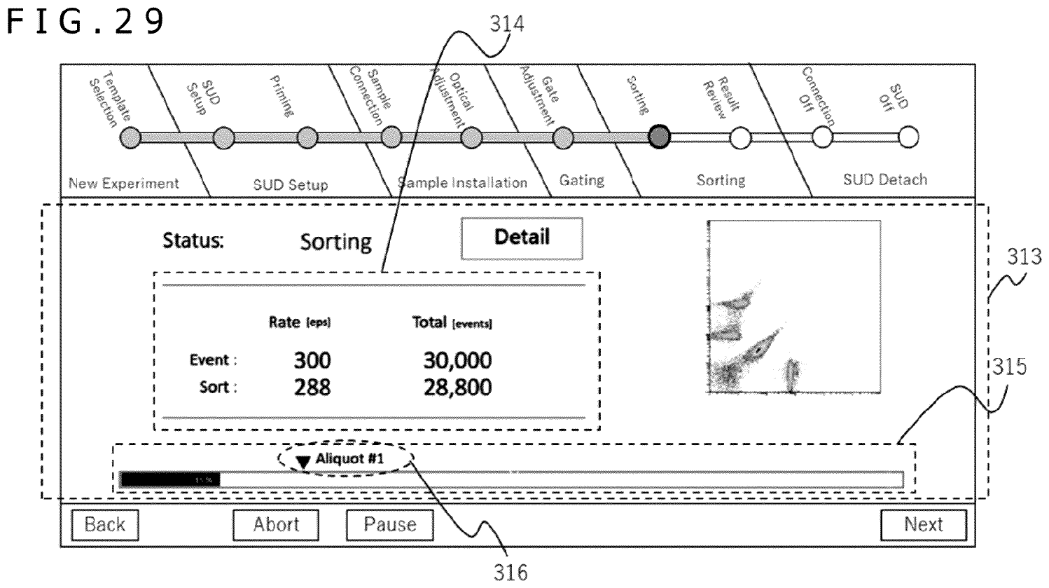

After the gate adjustment process completes, the bioparticle sorting apparatus advances the processing to a sorting process. As illustrated in FIG. 29, the sorting process, for example, may include a sorting step (displayed as “Sorting” in the same figure) for executing sorting of bioparticles and a result confirmation step (displayed as “Result Review” in the same figure) for confirming a sorting result.

In addition, partway through the sorting step, the bioparticle sorting apparatus may, in reference to the operation control data (specifically, aliquot setting data), execute an aliquot obtainment step for checking that the bioparticles being sorted are bioparticles that are set as an objective. By checking in the aliquot obtainment step, the second user can check whether bioparticles set as an objective are being sorted and, in a case where bioparticles set as an objective are not being sorted, can, by interrupting the sorting process or adjusting a processing condition, prevent a precious sample from being wastefully consumed. For the purpose of this checking, analysis by another bioparticle analysis apparatus may be performed, as described above.

In the sorting step, the information processing unit 102 causes a screen 313 that indicates a sorting status, to be displayed in the fourth display area, as illustrated in the same figure. As illustrated in the same figure, this screen may display an area 314 for displaying the number of events and/or an event obtainment speed (eps) as well as the number of sorts and/or a sorting speed (eps).

The information processing unit 102 may cause this screen to display an image 315 (a bar is displayed as this image in the same figure) that indicates a progress status for a sorting operation. An image 316 indicating a timing at which an aliquot obtainment step is executed may be added to the bar. As a result, the second user can know that a timing for executing the aliquot obtainment step has arrived and can perform preparatory work or other work, for example, up until this timing, and it is possible to improve convenience for the second user. The information processing unit 102 may display an image indicating the timing in reference to control data inputted in the first display mode, specifically, in reference to the aliquot setting data.

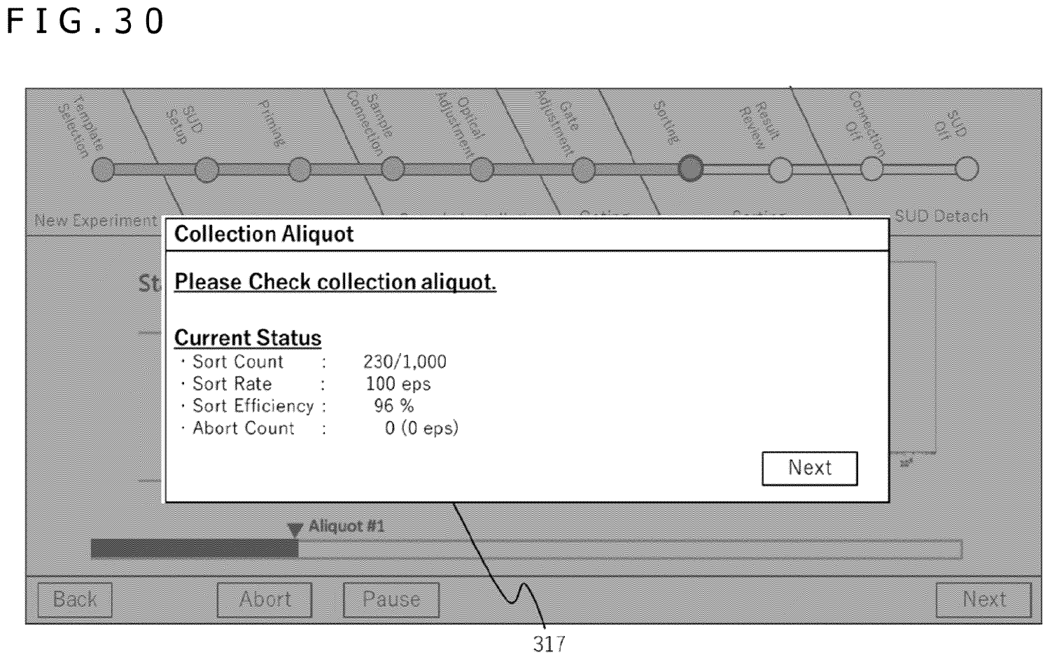

Once the timing for executing the aliquot obtainment step has arrived, the bioparticle sorting apparatus 100 pauses the sorting process and, for example, causes a window 317 that is illustrated in FIG. 30 to be displayed. As illustrated in the same figure, this window may display a checking technique for an aliquot and/or the current sorting status. In addition, this window displays a button for advancing processing forward.

In response to the button being selected, the information processing unit 102 displays a screen for prompting the second user to select whether to continue the sorting process. This screen includes a continue button for causing the information processing unit 102 to continue the sorting process and a stop button for stopping the sorting process. In response to the second user selecting the continue button, the information processing unit 102 continues the sorting process. In response to the second user selecting the stop button, the bioparticle sorting apparatus 100 stops the sorting process. Note that this screen may include a readjustment button for performing a gate adjustment again. In response to the second user selecting the readjustment button, the bioparticle sorting apparatus 100 returns the processing to the gate adjustment process.

In the result checking step, the bioparticle sorting apparatus 100 causes a screen for indicating a sorting result to be displayed in the fourth display area. This screen may display the number of events and/or the number of sorts.

### (End Process)

After the sorting process completes, the bioparticle sorting apparatus 100 advances the processing to an end process. As illustrated in FIG. 24, the end process, for example, may include a recovery container removing step (displayed as “Collection Off” in the same figure) for recovering a container that includes sorted bioparticles from the bioparticle sorting apparatus 100 and a chip removing step (displayed as “SUD Off”) for removing a microchip used for sorting from the bioparticle sorting apparatus 100. A screen displayed in the fourth display area for the purpose of these steps may be selected, as appropriate, by a person skilled in the art.

A sorting process is executed in the second display mode in accordance with processing as above.

### (3) Variations

In (1) and (2) above, description is given in relation to a bioparticle sorting apparatus that executes a sorting process on bioparticles (specifically, bioparticles flowing aligned in a row within a flow path) included in a biological sample supplied to a flow path, but the present disclosure may be applied to a bioparticle sorting apparatus that executes a sorting process on a group of bioparticles present two-dimensionally or three-dimensionally. In other words, a bioparticle sorting apparatus according to the present disclosure may be configured to execute a sorting process on a group of bioparticles present two-dimensionally or three-dimensionally. As a bioparticle sorting apparatus configured in such a manner, for example, it is possible to give a bioparticle sorting apparatus that executes a sorting process on a group of bioparticles present within a well disposed two-dimensionally or three-dimensionally, a bioparticle sorting apparatus that executes a sorting process on a group of bioparticles present two-dimensionally or three-dimensionally on any support body (for example, a cell culture surface or a cell immobilizing surface), or a bioparticle sorting apparatus that executes a bioparticle sorting process on a group of bioparticles (for example, biological tissue, etc.) that forms a three-dimensional structure.

As an example of a bioparticle sorting apparatus that executes a sorting process on a group of bioparticles present in a well, it is possible to give a bioparticle sorting apparatus that executes a bioparticle sorting process using a particle capturing chip described in Japanese Patent Laid-open No. 2020-174598.

As an example of a bioparticle sorting apparatus that executes a sorting process on a group of bioparticles present two-dimensionally on any surface, it is possible to give a bioparticle sorting apparatus that sorts specific bioparticles from among a group of bioparticles (cells) that is immobilized to a certain surface via a linker that can decompose (for example, a photodegradable linker, etc.).

These apparatuses, for example, may be configured to obtain a fluorescence image or a fluorescence signal for bioparticles with use of an optical detector that includes a microscope, etc., and, according to the obtained fluorescence signal or fluorescence image, specify bioparticles that should be sorted.

These apparatuses may be configured to extract only bioparticles specified in such a manner from a well, or may be configured to release (for example, cutting linkers that secure these bioparticles) immobilization for only bioparticles specified in such a manner.

These apparatuses may be configured to have the first display mode and the second display mode described in (1) and (2) above. For example, in the first display mode, operation control data for specifying bioparticles that should be sorted is inputted. In addition, in the second display mode, a processing execution operation screen generated in reference to this operation control data is displayed.

### 3. Second Embodiment (Bioparticle Sorting System) of the Present Disclosure

The present disclosure provides a bioparticle sorting system that includes a first information processing apparatus that operates in a first display mode for accepting input of operation control data pertaining to a bioparticle sorting process and a second information processing apparatus that operates in second display mode in which a processing execution operation screen is displayed.

This bioparticle sorting system also includes a bioparticle sorting apparatus that is operated by the first information processing apparatus or the second information processing apparatus. This bioparticle sorting apparatus may have the configuration described in 2. above.

In addition, the present disclosure also provides the first information processing apparatus included in the bioparticle sorting system. The first information processing apparatus, for example, may include a display unit that is capable of touch input and displays a processing condition setting screen for accepting input of operation control data pertaining to a sorting process for a bioparticle-containing sample. This processing condition setting screen may be as described in 2. above, and description thereof also applies to the present embodiment.

In addition, the present disclosure also provides the second information processing apparatus included in the bioparticle sorting system.

The second information processing apparatus, for example, may include a display unit that is capable of touch input and displays a processing execution operation screen. This processing execution operation screen may be as described in 2. above, and description thereof also applies to the present embodiment.

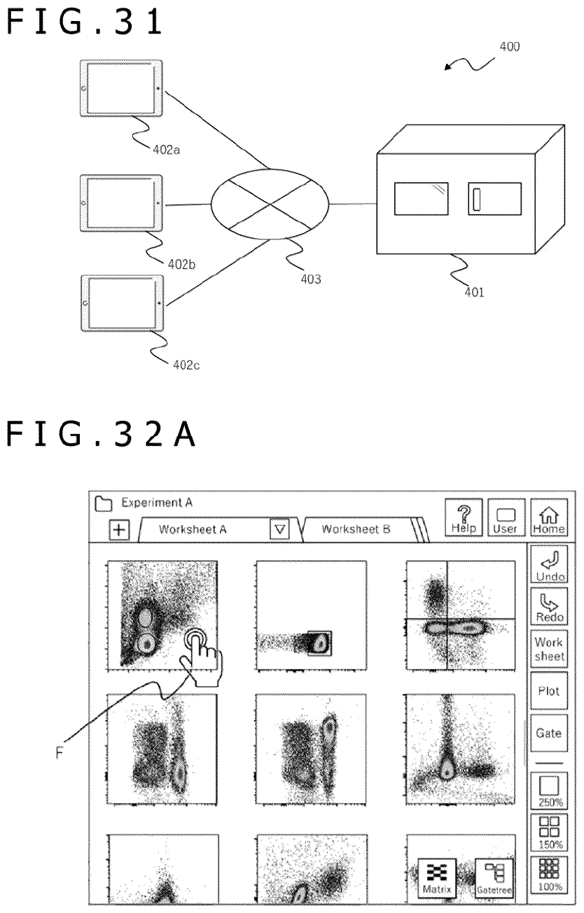

With reference to FIG. 31, description is given regarding an example of a bioparticle sorting system according to the present disclosure. The same figure illustrates an example of the configuration of a bioparticle sorting system according to the present disclosure. A bioparticle sorting system 400 illustrated in the same figure includes a bioparticle sorting apparatus 401 that executes a bioparticle sorting process and multiple information processing apparatuses 402a through 402c that are configured to be able to operate this bioparticle sorting apparatus. The number of information processing apparatuses included in the same system is not limited to three as illustrated in the same figure, and may be one or more. These multiple information processing apparatuses are connected to the bioparticle sorting apparatus via a network 403, for example.

Description is given below regarding a case in which the information processing apparatus 402a illustrated in the same figure operates in the first display mode described above and the information processing apparatus 402b operates in the second display mode described above.

First, the information processing apparatus 402a executes the steps S10, S11, and S12 described in 2.(2-1) above. Next, the information processing apparatus 402a generates operation control data by executing processing in the first display mode described in 2.(2-2) above.

More specifically, the information processing apparatus 402a operates as the information processing unit 102 described in 2.(2-2) above. The information processing apparatus 402a causes a display unit, which is attached to the apparatus or connected to the apparatus, to output the processing condition setting screen described in 2.(2-2) above, and accepts input of the operation control data via this screen. In addition, a sorting process by the bioparticle sorting apparatus 401 may be performed in the generation of the operation control data.

The information processing apparatus 402a transmits the generated operation control data to the bioparticle sorting apparatus 401.

Next, the information processing apparatus 402b executes the steps S10, S11, and S12 described in 2.(2-1) above. Next, the information processing apparatus 402b receives the operation control data from the bioparticle sorting apparatus 401. Then, the information processing apparatus 402b, in reference to the operation control data, executes processing in the second display mode described in 2.(2-3) above, and causes the bioparticle sorting apparatus 401 to execute a sorting process.

More specifically, the information processing apparatus 402b operates as the information processing unit 102 described in 2.(2-3) above. The information processing apparatus 402b causes a display unit attached to this apparatus or connected to this apparatus to output the processing execution operation screen described in 2.(2-3) above and, via this screen, causes the bioparticle sorting apparatus 401 to execute a sorting process.

Using a series of procedures as above, sorting of bioparticles is performed.

Note that the present disclosure can also take a configuration such as the following.

[1]

A bioparticle sorting apparatus including:
a display unit that is capable of touch input and displays a processing condition setting screen for accepting input of operation control data pertaining to a sorting process for a bioparticle-containing sample,
in which the processing condition setting screen includes a first display area for displaying an operation flow for setting a sorting process condition for the bioparticle-containing sample and a second display area for displaying measurement data pertaining to the bioparticle-containing sample, and
the first display area is disposed in an end section of the processing condition setting screen.
   [2]

The bioparticle sorting apparatus according to [1], in which
the first display area includes
   a procedure button area in which multiple processing buttons included in the operation flow are arranged following an order for processing, and
   a setting information display area that, in response to any processing button being touched, displays setting information corresponding to the processing button.
      [3]

The bioparticle sorting apparatus according to [2], in which
the first display area is disposed in a left-side end section of the processing condition setting screen, and the procedure button area is disposed in a left-side end section within the first display area, or
the first display area is disposed in a right-side end section of the processing condition setting screen, and the procedure button area is disposed in a right-side end section within the first display area.
   [4]

The bioparticle sorting apparatus according to any one of [1] to [3], in which,
in a case where the first display area is disposed in the left-side end section in the processing condition setting screen, the second display area is disposed in the right-side end section in the processing condition setting screen, and,
in a case where the first display area is disposed in the right-side end section in the processing condition setting screen, the second display area is disposed in the left-side end section in the processing condition setting screen.
   [5]

The bioparticle sorting apparatus according to any one of [1] to [4], in which
the second display area is configured to accept, according to a touch operation, input of gate information for identifying bioparticles to be sorted in the process.
   [6]

The bioparticle sorting apparatus according to any one of [1] to [5], in which
the second display area is configured to be able to display one or more plots, and,
in response to any plot being touched, an operation panel for performing an operation on the plot is caused to be displayed near the touched position.
   [7]

The bioparticle sorting apparatus according to [6], in which the operation panel includes one or more operation tool selection buttons.

[8]

The bioparticle sorting apparatus according to any one of [1] to [7], in which
the second display area is configured to be able to display one or more plots, and
the second display area causes one or more operation buttons for displaying data associated with the one or more plots to be displayed.
   [9]

The bioparticle sorting apparatus according to [8], in which the associated data is back data used to generate the plot.

[10]

The bioparticle sorting apparatus according to [9], in which the back data includes statistic information for each gate.

[11]

The bioparticle sorting apparatus according to [8], in which the associated data is fluorescence correction matrix data.

[12]

The bioparticle sorting apparatus according to any one of [9] to [11], in which it is possible to change the plot in response to an operation with respect to the back data.

[13]

The bioparticle sorting apparatus according to [12], in which
the second display area is configured to be able to display one or more plots, and,
in response to an axis of any plot being touched, a slider bar for changing a display format for the axis is caused to be displayed.
   [14]

The bioparticle sorting apparatus according to [13], in which the slider bar is for adjusting a numerical range or a scale for each axis.

[15]

The bioparticle sorting apparatus according to any one of [1] to [14], in which
the second display area is configured to be able to display one or more plots, and,
in response to a data display portion of any plot being touched, an enlarged image of the touched portion is displayed.
   [16]

The bioparticle sorting apparatus according to any one of [1] to [15], in which
the second display area includes an operation button area that includes a plot button for performing an operation on a plot displayed within the area and/or a gate button for performing an operation on a gate within the plot.
   [17]

The bioparticle sorting apparatus according to any
one of [1] to [16], in which the display unit is disposed on any surface of a housing for the bioparticle sorting apparatus.
   [18]

An information processing apparatus including:
a display unit that is capable of touch input and displays a processing condition setting screen for accepting input of operation control data pertaining to a sorting process for a bioparticle-containing sample,
in which the processing condition setting screen includes a first display area for displaying an operation flow for setting a sorting process condition for the bioparticle-containing sample and a second display area for displaying measurement data pertaining to the bioparticle-containing sample, and
the first display area is disposed in an end section of the processing condition setting screen.

### REFERENCE SIGNS LIST

100: Bioparticle sorting apparatus
101: Display unit
102: Information processing unit

1. A bioparticle sorting apparatus comprising:
a display unit that is capable of touch input and displays a processing condition setting screen for accepting input of operation control data pertaining to a sorting process for a bioparticle-containing sample,
wherein the processing condition setting screen includes a first display area for displaying an operation flow for setting a sorting process condition for the bioparticle-containing sample and a second display area for displaying measurement data pertaining to the bioparticle-containing sample, and
the first display area is disposed in an end section of the processing condition setting screen.

2. The bioparticle sorting apparatus according to claim 1, wherein
the first display area includes
a procedure button area in which multiple processing buttons included in the operation flow are arranged following an order for processing, and
a setting information display area that, in response to any processing button being touched, displays setting information corresponding to the processing button.

3. The bioparticle sorting apparatus according to claim 2, wherein
the first display area is disposed in a left-side end section of the processing condition setting screen, and the procedure button area is disposed in a left-side end section within the first display area, or
the first display area is disposed in a right-side end section of the processing condition setting screen, and the procedure button area is disposed in a right-side end section within the first display area.

4. The bioparticle sorting apparatus according to claim 1, wherein,
in a case where the first display area is disposed in a left-side end section in the processing condition setting screen, the second display area is disposed in a right-side end section in the processing condition setting screen, and,
in a case where the first display area is disposed in a right-side end section in the processing condition setting screen, the second display area is disposed in a left-side end section in the processing condition setting screen.

5. The bioparticle sorting apparatus according to claim 1, wherein
the second display area is configured to accept, according to a touch operation, input of gate information for identifying bioparticles to be sorted in the process.

6. The bioparticle sorting apparatus according to claim 1, wherein
the second display area is configured to be able to display one or more plots, and,
in response to any plot being touched, an operation panel for performing an operation on the plot is caused to be displayed near the touched position.

7. The bioparticle sorting apparatus according to claim 6, wherein the operation panel includes one or more operation tool selection buttons.

8. The bioparticle sorting apparatus according to claim 1, wherein
the second display area is configured to be able to display one or more plots, and
the second display area causes one or more operation buttons for displaying data associated with the one or more plots to be displayed.

9. The bioparticle sorting apparatus according to claim 8, wherein the associated data is back data used to generate the plot.

10. The bioparticle sorting apparatus according to claim 9, wherein the back data includes statistic information for each gate.

11. The bioparticle sorting apparatus according to claim 8, wherein the associated data is fluorescence correction matrix data.

12. The bioparticle sorting apparatus according to claim 9, wherein it is possible to change the plot in response to an operation with respect to the back data.

13. The bioparticle sorting apparatus according to claim 12, wherein
the second display area is configured to be able to display one or more plots, and,
in response to an axis of any plot being touched, a slider bar for changing a display format for the axis is caused to be displayed.

14. The bioparticle sorting apparatus according to claim 13, wherein the slider bar is for adjusting a numerical range or a scale for each axis.

15. The bioparticle sorting apparatus according to claim 1, wherein
the second display area is configured to be able to display one or more plots, and,
in response to a data display portion of any plot being touched, an enlarged image of the touched portion is displayed.

16. The bioparticle sorting apparatus according to claim 1, wherein
the second display area includes an operation button area that includes a plot button for performing an operation on a plot displayed within the area and/or a gate button for performing an operation on a gate within the plot.

17. The bioparticle sorting apparatus according to claim 1, wherein the display unit is disposed on any surface of a housing for the bioparticle sorting apparatus.

18. An information processing apparatus comprising:
a display unit that is capable of touch input and displays a processing condition setting screen for accepting input of operation control data pertaining to a sorting process for a bioparticle-containing sample,
wherein the processing condition setting screen includes a first display area for displaying an operation flow for setting a sorting process condition for the bioparticle-containing sample and a second display area for displaying measurement data pertaining to the bioparticle-containing sample, and
the first display area is disposed in an end section of the processing condition setting screen.
